(19) United States
(12) Patent Application Publication
(10) Pub. No.: US 2021/0302440 A1
(43) Pub. Date: Sep. 30, 2021

(54) ADRENOMEDULLIN (ADM) FOR DIAGNOSIS AND/OR PREDICTION OF DEMENTIA AND ANTI-ADRENOMEDULLIN BINDER FOR USE IN THERAPY OR PREVENTION OF DEMENTIA

(71) Applicant: SPHINGOTEC GMBH, Hennigsdorf (DE)

(72) Inventor: Olle MELANDER, Limhamm (SE)

(73) Assignee: SPHINGOTEC GMBH, Hennigsdorf (DE)

(21) Appl. No.: 16/968,483

(22) PCT Filed: Feb. 7, 2019
(86) PCT No.: PCT/EP2019/052982

§ 371 Date: Aug. 7, 2020

(30) Foreign Application Priority Data
Feb. 8, 2018 (EP) 18155682.0

(51) Int. Cl. G01N 33/68; C07K 16/22

(52) U.S. Cl. CPC G01N 33/6896; C07K 16/22; G01N 2333/475; G01N 2800/52; G01N 2800/2814

(57) ABSTRACT

Subject matter of the present invention is a method for diagnosing dementia, or determining the risk of getting dementia in a subject that does not have dementia, or monitoring therapy or monitoring or guiding intervention in a subject that has dementia, or monitoring therapy or monitoring or guiding preventive intervention in a subject that is at risk of getting dementia.

Subject matter of the present invention is a method for:
a) diagnosing dementia, or
b) determining the risk of getting dementia in a subject that does not have dementia, or
c) monitoring therapy or monitoring or guiding intervention in a subject that has dementia, or
d) monitoring therapy or monitoring or guiding preventive intervention in a subject that is at risk of getting dementia,
   wherein the level of mature ADM-NH₂ according to SEQ ID No.: 4 is determined in a sample of bodily fluid of a subject and wherein said level of mature ADM-NH₂ is compared with a threshold level,
   and wherein
a) said subject is diagnosed with dementia if the level of mature ADM-NH₂ according to SEQ ID No.: 4 is below said threshold level, or wherein
b) said subject has an enhanced risk of getting dementia if said level of mature ADM-NH₂ according to SEQ ID No.: 4 is below said threshold level, or wherein
c) the status of a subject having dementia or being at risk of getting dementia is improving under therapy or intervention if said level of mature ADM-NH₂ according to SEQ ID No.: 4 is increased during the course of therapy or intervention and/or wherein intervention maybe continued if said level of mature ADM-NH₂ according to SEQ ID No.: 4 is increased above said level threshold.

Another subject matter of the present invention is a method for:
a) diagnosing dementia, or
b) determining the risk of getting dementia in a subject that does not have dementia, or
c) monitoring therapy or monitoring or guiding intervention in a subject that has dementia, or
d) monitoring preventive therapy or monitoring or guiding preventive intervention in a subject that is at risk of getting dementia,
   wherein a marker ratio is determined that maybe the ratio of the level of mature ADM-NH₂ according to SEQ ID No.: 4 determined in a sample of bodily fluid of said subject to the level of pro-Adrenomedullin or a fragment thereof (which is not mature ADM-NH₂ according to SEQ ID No.: 4) determined in a sample of bodily fluid of said subject and wherein said marker ratio is compared to a threshold ratio
   and wherein
a) said subject is diagnosed with dementia if the marker ratio ADM-NH₂/pro-Adrenomedullin or a fragment thereof is below said ratio threshold, or wherein
b) said subject has an enhanced risk of getting dementia if the marker ratio pro-Adrenomedullin or a fragment thereof is below said ratio threshold, or wherein
c) the status of a subject having dementia or being at risk of getting dementia is improving under therapy or intervention if said marker ratio is increased during the course of therapy or intervention and wherein intervention maybe continued if said level of the marker ratio is increased above said ratio threshold.

Alternatively, the level of mature ADM-NH₂ according to SEQ ID No.: 4 determined in a sample of bodily fluid of said subject and the level of pro-Adrenomedullin or a fragment thereof (which is not mature ADM-NH₂ according to SEQ ID No.: 4) determined in a sample of bodily fluid of said subject will be combined by a mathematical algorithm, wherein the result of said algorithm is used for diagnosing dementia, or determining the risk of getting dementia in a subject that does not have dementia, or monitoring therapy or monitoring or guiding intervention in a subject that has dementia, or monitoring preventive therapy or monitoring or guiding preventive intervention in a subject that is at risk of getting dementia.

Dementia is a clinical syndrome characterized by a cluster of symptoms and signs manifested by difficulties in memory, disturbances in language, psychological and psychiatric changes, and impairments in activities of daily living. The different causes (sometimes referred to as subtyping) of dementia syndrome are: Alzheimer's disease (about 50% of cases), vascular dementia (about 25%), mixed Alzheimer's disease and vascular dementia (included in the above, 25%), Lewy body dementia (15%) and others (about 5% combined) including frontotemporal dementia, focal dementias (such as progressive aphasia), subcortical dementias (such as Parkinson's disease dementia), and secondary causes of dementia syndrome (such as intracranial lesions).

Alzheimer's disease (AD) is the most prevalent form of dementia. AD is increasing rapidly in frequency as the world's population ages and more people enter the major risk period for this age-related disorder. From the 5.3 million US citizens affected now, the number of victims will increase to 13 million or more by 2050; worldwide the total number of affected individuals will increase to a staggering 100 million (Alzheimer's Association. 2015 Alzheimer's disease facts and figures. Alzheimers Dement 2015; 11:332-84). Key molecular mechanisms and histopathological hallmarks in the AD brain comprise a dynamic cascade of biochemical events including the pathological amyloidogenic cleavage of the amyloid precursor protein (APP), the generation of various beta-amyloid species including the amyloid-beta peptide (Aβ₁₋₄₂), dimers, trimers, oligomers and subsequent amyloid aggregation and deposition in plaques, abnormal hyperphosphorylation and aggregation of tau protein, progressive intracellular neurofibrillary degeneration, changes within the innate immune system and inflammation.

About 5% of patients develop symptoms before age 65 and are characterized as patients with “early-onset Alzheimer's disease” (EOAD). Most of these patients have the sporadic form of the disease, but 10-15% have a genetic form that is generally inherited as an autosomal dominant fashion. Three genes have been suggested to be involved in the development of EOAD: Presenilin 1 and 2 and the amyloid precursor protein (APP) gene. Other candidate genes are also under investigation. Genetic forms tend to start at age 30 or 40 and have an aggressive course while sporadic EOAD tend to start after age 50 and have, in general, a temporal profile similar to the “late onset Alzheimer's disease” (LOAD) one.

Mental status testing evaluates memory, ability to solve simple problems and other thinking skills. Such tests give an overall sense of whether a person is aware of symptoms, knows the date, time, and where he or she is, can remember a short list of words, follow instructions and do simple calculations. The mini-mental state exam (MMSE) and the mini-cog test are two commonly used tests. The MMSE or Folstein test is a 30-point questionnaire that is used extensively in clinical and research settings to measure cognitive impairment (Pangman, et al. 2000. Applied Nursing Research 13 (4): 209-213; Folstein et al. 1975. Journal of Psychiatric Research. 12 (3): 189-98). During the MMSE, a health professional asks a patient a series of questions designed to test a range of everyday mental skills. The maximum MMSE score is 30 points. A score of 20 to 24 suggests mild dementia, 13 to 20 suggests moderate dementia, and less than 12 indicates severe dementia. On average, the MMSE score of a person with Alzheimer's declines about two to four points each year. Advantages to the MMSE include requiring no specialized equipment or training for administration, and has both validity and reliability for the diagnosis and longitudinal assessment of Alzheimer's disease. During the mini-cog, a person is asked to complete two tasks, remember and a few minutes later repeat the names of three common objects and draw a face of a clock showing all 12 numbers in the right places and a time specified by the examiner. The results of this brief test can help a physician determine if further evaluation is needed. Other tests are also used, such as the Hodkinson abbreviated mental test score (Hodkinson 1972. Age and ageing. 1 (4): 233-8) or the General Practitioner Assessment of Cognition, computerized tests such as CoPs and Mental Attributes Profiling System as well as longer formal tests for deeper analysis of specific deficits.

Mild cognitive impairment (MCI) is a heterogeneous clinical condition with several underlying causes. However, the large proportion of MCI represents a transitional state between healthy aging and very mild AD (DeCarli 2003. Lancet Neurol. 2:15-21). Accordingly, studies suggest that MCI subjects tend to progress to clinically probable AD at a rate of approximately 10%-15% per year (Markesbery 2010. J Alzheimers Dis. 19:221-228).

Alzheimer's disease is usually diagnosed based on the person's medical history, history from relatives, and behavioral observations. The presence of characteristic neurological and neuropsychological features and the absence of alternative conditions is supportive. Advanced medical imaging with computed tomography (CT) or magnetic resonance imaging (MRI), and with single-photon emission computed tomography (SPECT) or positron emission tomography (PET) can be used to help exclude other cerebral pathology or subtypes of dementia. Moreover, it may predict conversion from prodromal stages (mild cognitive impairment) to Alzheimer's disease. Assessment of intellectual functioning including memory testing can further characterize the state of the disease. Medical organizations have created diagnostic criteria to ease and standardize the diagnostic process for practicing physicians. The diagnosis can be confirmed with very high accuracy post-mortem when brain material is available and can be examined histologically.

To date, only symptomatic treatments exist for this disease, all trying to counterbalance the neurotransmitter disturbance. Three cholinesterase inhibitors are currently available and have been approved for the treatment of mild to moderate AD. A further therapeutic option available for moderate to severe AD is memantine, an N-methyl-D-aspartate receptor noncompetitive antagonist. Treatments capable of stopping or at least effectively modifying the course of AD, referred to as ‘disease-modifying’ drugs, are still under extensive research.

New therapies are urgently needed to treat affected patients and to prevent, defer, slow the decline, or improve the symptoms of AD. It has been estimated that the overall frequency of the disease would be decreased by nearly 50% if the onset of the disease could be delayed by 5 years. Symptomatic treatments are drugs aimed at cognitive enhancement or control of neuropsychiatric symptoms and typically work through neurotransmitter mechanisms; disease-modifying therapies or treatments (DMTs) are agents that prevent, delay, or slow progression and target the underlying pathophysiologic mechanisms of AD. Currently there are more than 100 agents in the AD treatment development pipeline (Cummings et al. 2017. Alzheimer's & Dementia: Translational Research & Clinical Interventions 3: 367-384).

Dementia with Lewy bodies (DLB) is a type of dementia that worsens over time. Additional symptoms may include fluctuations in alertness, visual hallucinations, slowness of movement, trouble walking, and rigidity. DLB is the most common cause of dementia after Alzheimer's disease and vascular dementia. It typically begins after the age of 50. About 0.1% of those over 65 are affected. Men appear to be more commonly affected than women. The underlying mechanism involves the formation of Lewy bodies in neurons, consisting of alpha-synuclein protein. A diagnosis may be suspected based on symptoms, with blood tests and medical imaging done to rule out other possible causes. At present no cure for DLB exists. Treatments are supportive and attempt to relieve some of the motor and psychological symptoms associated with the disease. Acetylcholinesterase inhibitors, such as donepezil, may provide some benefit. Some motor problems may improve with levodopa. For review see McKeith et al. 2017. Neurology 89: 88-100.

Vascular dementia (VaD), also known as multi-infarct dementia (MID) and vascular cognitive impairment (VCI), is dementia caused by problems in the supply of blood to the brain, typically a series of minor strokes, leading to worsening cognitive decline that occurs step by step. The term refers to a syndrome consisting of a complex interaction of cerebrovascular disease and risk factors that lead to changes in the brain structures due to strokes and lesions, and resulting changes in cognition. The temporal relationship between a stroke and cognitive deficits is needed to make the diagnosis. Differentiating the different dementia syndromes can be challenging, due to the frequently overlapping clinical features and related underlying pathology. In particular, Alzheimer's dementia often co-occurs with vascular dementia. People with vascular dementia present with progressive cognitive impairment, acutely or sub-acutely as in mild cognitive impairment, frequently step-wise, after multiple cerebrovascular events (strokes). For review see Venkat et al. 2015. Exp Neurol 272: 97-108.

Frontotemporal dementia (FTD) is the clinical presentation of frontotemporal lobar degeneration, which is characterized by progressive neuronal loss predominantly involving the frontal or temporal lobes, and typical loss of over 70% of spindle neurons, while other neuron types remain intact. FTD accounts for 20% of young-onset dementia cases. Signs and symptoms typically manifest in late adulthood, more commonly between the ages of 55 and 65, approximately equally affecting men and women. Common signs and symptoms include significant changes in social and personal behavior, apathy, blunting of emotions, and deficits in both expressive and receptive language. Currently, there is no cure for FTD, but there are treatments that help alleviate symptoms. For review see Bott et al. 2014. Neurodegener Dis Manag 4(6): 439-454.

The peptide adrenomedullin (ADM) was described for the first time in Kitamura et al. (Kitamura et al. 1993. Biochemical and Biophysical Research Communications 192 (2): 553-560) as a novel hypotensive peptide comprising 52 amino acids, which had been isolated from a human pheochromocytoma. In the same year, cDNA coding for a precursor peptide comprising 185 amino acids and the complete amino acid sequence of this precursor peptide were also described. The precursor peptide, which comprises, inter alia, a signal sequence of 21 amino acids at the N-terminus, is referred to as “preproadrenomedullin” (pre-proADM). Pre-proADM comprises 185 amino acids (SEQ ID No.: 1). The mature ADM-NH₂ is displayed in SEQ ID No. 4 and ADM-Gly is displayed in SEQ No. 5.

The mature adrenomedullin peptide is an amidated peptide (ADM-NH₂), which comprises 52 amino acids (SEQ ID No: 4) and which comprises the amino acids 95 to 146 of pre-proADM, from which it is formed by proteolytic cleavage. To date, substantially only a few fragments of the peptide fragments formed in the cleavage of the pre-proADM have been more exactly characterized, in particular the physiologically active peptides adrenomedullin (ADM) and “PAMP”, a peptide comprising 20 amino acids (22-41) (SEQ ID No.: 2), which follows the 21 amino acids of the signal peptide in pre-proADM. Furthermore, for both, ADM and PAMP, physiologically active sub-fragments were discovered and investigated in more detail. The discovery and characterization of ADM in 1993 triggered intensive research activity and a flood of publications, the results of which have recently been summarized in various review articles, in the context of the present description, reference being made in particular to the articles Takahashi 2001. Peptides 22: 1691; Eto et al. 2001. Peptides 22: 1693-1711 and Hinson et al. 2000 Endocrine Reviews 21(2): 138-167.

In the scientific investigations to date, it has been found, inter alia, that ADM may be regarded as a polyfunctional regulatory peptide. It is released into the circulation partially in an inactive form extended by glycine (Kitamura et al. 1998. Biochem. Biophys. Res. Commun. 244(2): 551-555). There is also a binding protein (Pio et al. 2001. The Journal of Biological Chemistry 276(15): 12292-12300), which is specific for ADM and probably likewise modulates the effect of ADM.

Those physiological effects of ADM as well as of PAMP, which are of primary importance in the investigations to date, were the effects influencing blood pressure. Thus, ADM is an effective vasodilator.

It has furthermore been found that the above-mentioned further physiologically active peptide PAMP formed from pre-proADM likewise exhibits a hypotensive effect, even if it appears to have an action mechanism differing from that of ADM (Eto et al. 2001. Peptides 22: 1693-1711; Hinson et al. 2000 Endocrine Reviews 21(2):138-167; Kuwasako et al. 1997. FEBS Lett 414(1): 105-110; Kuwasaki et al. 1999. Ann. Clin. Biochem. 36: 622-628; Tsuruda et al. 2001. Life Sci. 69(2): 239-245; Kangawa et al. EP 0 622 458).

Furthermore, it was found that the concentrations of ADM, which can be measured in the circulation and other biological fluids, are in a number of pathological states, significantly above the concentrations to be found in healthy control persons. Thus, the ADM level in patients with congestive heart failure, myocardial infarction, kidney diseases, hypertensive disorders, diabetes mellitus, in the acute phase of shock and in sepsis and septic shock are significantly increased, although to different extents. The PAMP concentrations are also increased in some of said pathological states, but the plasma levels are reduced relative to ADM (Eto et al. 2001. Peptides 22: 1693-1711).

Furthermore, it is known that unusual high concentrations of ADM are to be observed in sepsis or in septic shock (Eto et al. 2001. Peptides 22: 1693-1711; Hirata et al. 1996. Journal of Clinical Endocrinology and Metabolism 81(4): 1449-1453; Ehlenz et al. 1997. Exp Clin Endocrinol Diabetes 105: 156-162; Tomoda et al. 2001. Peptides 22: 1783-1794; Ueda et al. 1999 Am. J. Respir. Crit. Care Med. 160: 132-136; Wang et al. 2001. Peptides 22: 1835-1840). The findings are related to the typical hemodynamic changes which are known as typical phenomena of the course of a disease in patients with sepsis and other severe syndromes, such as, for example, SIRS. Adrenomedullin plays pivotal roles during sepsis development (Wang, Shock 1998, 10(5):383-384; Wang et al. 1998. Archives of surgery 133(12): 1298-1304) and in numerous acute and chronic diseases (Parlapiano et al. 1999. European Review for Medical and Pharmacological Sciences 3:53-61; Hinson et al. 2000 Endocrine Reviews 21(2):138-167).

Several methods were described to measure circulating levels of ADM: either ADM directly or indirectly by determining a more stable fragment of its cognate precursor peptide. Recently a method was published, describing an assay to measure circulating mature ADM (Weber et al. 2017. Journal of applied Labaratory Medicine, 2(2): 222-233).

Other methods to quantify fragments derived from the ADM precursor have been described, e.g. the measurement of MR-proADM (Morgenthaler et al. 2005. Clin Chem 51(10):1823-9), PAMP (Washimine et al. 1994. Biochem Biophys Res Commun 202(2):1081-7) and CT-proADM (EP 2 111 552). A commercial homogeneous time-resolved fluoroimmunoassay for the measurement of MR-proADM in plasma on a fully automated system is available (BRAHMS MR-proADM KRYPTOR; BRAHMS GmbH, Hennigsdorf, Germany) (Caruhel et al. 2009. Clin Biochem 42(7-8):725-8). As these peptides are generated in a stoichiometric ratio from the same precursor, their plasma levels are correlated to a certain extent.

The role of MR-proADM in dementia and AD was explored in a few studies. Plasma levels of MR-proADM measured in patients with probable AD were increased compared to elderly cognitively normal healthy controls (Buerger et al. 2009. Biological Psychiatry 2009; 65:979-984). The blood concentration of MR-proADM alone showed a classification accuracy with a sensitivity of 47% at a specificity of 81% and the ratio of MR-proADM with another biomarker, CT-proET-1, showed a sensitivity of 66% at a specificity of 81% for the detection of AD. Moreover, plasma concentrations of MR-proADM have predictive value in the progression from predementia MCI to clinical AD (Buerger et al. 2010. J Clin Psychiatry 72(4): 556-563). MR-proADM was also measured in a population-based cohort of more than 5000 individuals without prevalent dementia and were shown to be elevated in participants who developed dementia, but indicated no increased risk after adjusting for traditional risk factors (Holm et al. 2017. Journal of Internal Medicine 282: 94-101). In patients participating in a longitudinal study on arteriosclerosis MR-proADM levels were significantly increased with cerebral deep white matter lesions (DWMLs) grade progression (Kuriyama et al. 2017. Journal of Alzheimer's Disease 56: 1253-1262). Moreover, a significant inverse correlation was observed between MR-proADM levels and cognitive test scores.

Adrenomedullin was shown to be increased in the frontal cortex from AD patients when compared to age-matched controls (Ferrero et al. 2017. Mol Neurobiol. doi: 10.1007/s12035-017-0700-6, E-Pub ahead of print). However, nothing is known about plasma ADM in patients with dementia, especially Alzheimer's disease.

A model of subcortical vascular dementia was reproduced in mice by placing microcoils bilaterally on the common carotid arteries. Using mice overexpressing circulating ADM, the effect of ADM was assessed on cerebral perfusion, cerebral angioarchitecture, oxidative stress, white matter change, cognitive function, and brain levels of cAMP, vascular endothelial growth factor, and basic fibroblast growth factor. These data indicate that ADM promotes arteriogenesis and angiogenesis, inhibits oxidative stress, preserves white matter integrity, and prevents cognitive decline after chronic cerebral hypoperfusion. Thus, ADM may serve as a strategy to tackle subcortical vascular dementia. (Maki et al. 2011. Stroke 42:1122-1128).

It was a surprising finding of the present invention that levels of mature ADM are significantly decreased in healthy patients that later develop dementia, in particular AD. Furthermore, it has been surprisingly found that levels of mature ADM are significantly decreased if a subject has dementia, in particular Alzheimer's dementia. It can be seen from the examples that baseline levels of Adrenomedullin, in particular ADM-NH₂ according to SEQ ID No.: 4 independently predicts the presence of dementia, in particular Alzheimer's dementia.

Furthermore, it has been surprisingly found that a subject is diagnosed with dementia, in particular AD, if the marker ratio ADM-NH₂/pro-Adrenomedullin or a fragment thereof is below a certain marker level ratio. Furthermore, it has been surprisingly found that a subject has an enhanced risk of getting dementia if the marker level ratio ADM-NH₂/pro-Adrenomedullin or a fragment thereof is below a certain marker level ratio threshold. Furthermore, it has been surprisingly found that the status of a subject having dementia, in particular AD, or being at risk of getting dementia, in particular AD, is improving under therapy or intervention if said marker level ratio is increased during the course of therapy or intervention and wherein intervention maybe continued if said level of the marker ratio is increased above said ratio threshold.

Alternatively, the level of mature ADM-NH₂ according to SEQ ID No.: 4 determined in a sample of bodily fluid of said subject and the level of pro-Adrenomedullin or a fragment thereof (which is not mature ADM-NH₂ according to SEQ ID No.: 4) determined in a sample of bodily fluid of said subject will be combined by a mathematical formula or algorithm, wherein the result of said formula or algorithm is used for diagnosing dementia, or determining the risk of getting dementia in a subject that does not have dementia, or monitoring therapy or monitoring or guiding intervention in a subject that has dementia, or monitoring preventive therapy or monitoring or guiding preventive intervention in a subject that is at risk of getting dementia.

It was, thus, the surprising finding of the invention that the level of mature ADM (mature ADM-NH₂ according to SEQ ID No.: 4) in the circulation is decreased in a subject having dementia or being at risk of getting dementia. Furthermore, the level of pro-Adrenomedullin or a fragment thereof (which is not mature ADM-NH₂ according to SEQ ID No.: 4) in the circulation is increased in a subject having dementia or being at risk of getting dementia. It is known that mature ADM (mature ADM-NH₂ according to SEQ ID No.: 4) is a hormone responsible for the vascular integrity and for the function of the vascular endothelium. It is further known that dysfunction of the vascular endothelium has been linked to a broad spectrum of the most dreadful human diseases, such as peripheral vascular disease, stroke, heart disease, diabetes, chronic kidney failure, and metastasis and dementia (Rajendran et al. 2013. Int. J. Biol. Sci. 9(19: 1057-1069).

High levels of pro-Adrenomedullin or a fragment thereof (which is not mature ADM-NH₂ according to SEQ ID No.: 4) in the circulation seem to indicate the need of the body to repair the function of the vascular endothelium and the need to support vascular integrity. However, low levels of mature ADM (mature ADM-NH₂ according to SEQ ID No.: 4) indicate, that despite of high levels of pro-ADM the conversion from ADM-Gly to mature ADM (mature ADM-NH₂ according to SEQ ID No.: 4) seems to be disturbed.

It is known that MR-proADM concentrations in the circulation of patients with Alzheimer's disease, patients with MCI or subjects who will develop AD are elevated (Buerger et al. 2009. Biological Psychiatry 2009; 65:979-984; Buerger et al. 2010. J Clin Psychiatry 72(4): 556-563; Holm et al. 2017. Journal of Internal Medicine 282: 94-101). This shows that the pathway of ADM synthesis is activated. Nothing, however, is said about the concentration of biologically active ADM. It has been shown by the present invention that the concentration of active ADM (bio-ADM) in the circulation is surprisingly lower in patients with AD and patients who will develop AD (Example 6). Moreover, there is increasing evidence that impairment of the cerebral microvasculature is a contributing factor in the pathophysiology of Alzheimer disease (Iadecola 2013. The pathobiology of vascular dementia. Neuron 2013; 80(4):844-866). Results of histologic evaluation and albumin sampling studies show that an increased permeability of the blood-brain barrier (BBB) is likely a key mechanism (Benarroch 2007. Neurovascular unit dysfunction: a vascular component of Alzheimer disease? Neurology 68(20):1730-1732). In recent studies it was demonstrated that global BBB leakage in patients with early AD is associated with cognitive decline (Nation et al. 2019. Nature Medicine https://doi.org/10.1038/s41591-018-0297-y; van de Haar et al. 2016 Radiology 281(2): 527-535). N-terminal anti-ADM-antibodies were shown to stabilize Adrenomedullin and induce an increase in circulating active ADM (Geven et al. 2018. Effects of humanized anti-adrenomedullin antibody Adrecizumab (HAM8101) on vascular barrier function and survival in rodent models of systemic inflammation and sepsis. Shock 50(6):648-654; Geven et al. 2018. Vascular effects of adrenomedullin and the anti-adrenomedullin antibody Adrecizumab in sepsis. Shock 50(2):132-140). The effect of inducing a rapid increase in bio-ADM in the blood of healthy patients is shown in Example 7 and FIG. 8. The increase of ADM in the circulation results in a beneficial effect on endothelial cells e.g. reduction of capillary leakage. For example, an N-terminal anti-ADM antibody (HAM8101, Adrecizumab) was shown to enhance endothelial barrier function in experimental models of systemic inflammation and sepsis (Geven et al. 2018. Effects of humanized anti-adrenomedullin antibody Adrecizumab (HAM8101) on vascular barrier function and survival in rodent models of systemic inflammation and sepsis. Shock 50(6):648-654). Therefore, an N-terminal ADM-binder, more specifically an N-terminal anti-ADM antibody can be applied to increase the bio-ADM concentration in the blood of patients with dementia or being at risk of developing dementia, especially patients with Alzheimer's dementia.

Thus, in summary low levels of ADM in the circulation, in particular bio-active ADM, in a patient in the need of the body to repair the function of the vascular endothelium and the need to support vascular integrity, may indicate that processing of ADM is disturbed in said patient. A patient in need of the body to repair the function of the vascular endothelium and the need to support vascular integrity may be characterized and identified by determining the level of mature ADM-NH₂ according to SEQ ID No.: 4 in a sample of bodily fluid of a subject and wherein said level of mature ADM-NH₂ is compared with a threshold level as outlined in the methods of the present invention or by determining a marker ratio that maybe the ratio of the level of mature ADM-NH₂ according to SEQ ID No.: 4 determined in a sample of bodily fluid of said subject to the level of pro-Adrenomedullin or a fragment thereof (which is not mature ADM-NH₂ according to SEQ ID No.: 4) determined in a sample of bodily fluid of said subject and wherein said marker ratio is compared to a threshold ratio as outlined in the methods of the present invention.

Additionally or alternatively, a patient in need of the body to repair the function of the vascular endothelium and the need to support vascular integrity may be a patient with global BBB leakage or BBB breakdown. Global BBB leakage or BBB breakdown may be determined as follows: Measurement of the cerebrospinal fluid (CSF)/serum ratio of albumin or immunoglobulin G (IgG) (Akaishi et al. 2015. Neurology and Clinical Neuroscience 3: 94-100) or imaging techniques, e.g. dynamic susceptibility contrast enhanced magnetic resonance imaging (DSC-MRI) or dynamic contrast enhanced MRI (DCE-MRI) (Raja et al. 2018. Neuropharmacology 134: 259-271).

Thus, stratification and identification of patients in need of enhancing the levels of bio-ADM in order to prevent or prevent progress in human cognitive dysfunction or in order to prevent or treat dementia is performed by any of the methods as described above.

It has been shown that administration of N-terminal anti-ADM antibody a rapid increase in bio-ADM in the blood of healthy patients is shown in Example 7 and FIG. 8 that may help to repair the leaky or damaged blood brain barrier. Therefore, it seems plausible that administration of N-terminal anti-ADM antibody helps in the prevention and therapy of dementia in a subject that is identified and/or stratified as described above.

Therefore, it is another aim to provide a therapy in subjects having decreased levels of mature ADM (mature ADM-NH₂ according to SEQ ID No.: 4) and/or having a decreased ratio of the level of mature ADM-NH₂ according to SEQ ID No.: 4 determined in a sample of bodily fluid of said subject to the level of pro-Adrenomedullin or a fragment thereof (which is not mature ADM-NH₂ according to SEQ ID No.: 4) determined in a sample of bodily fluid. Said patient group maybe treated with an Anti-adrenomedullin (ADM) antibody or an anti-adrenomedullin antibody fragment or anti-ADM non-Ig scaffold for use in prevention and therapy of dementia in a subject, wherein said anti-ADM antibody or anti-ADM fragment or anti-ADM non-Ig scaffold binds to the N-terminal part (aa 1-21) of adrenomedullin:

(SEQ ID No. 21)
YRQSMNNFQGLRSFGCRFGTC.

In another embodiment of the present invention said subject to be treated shows in addition to the above-mentioned criteria signs of mild cognitive impairments or signs of dementia.

It is known that the administration of Anti-adrenomedullin (ADM) antibody or an anti-adrenomedullin antibody fragment or anti-ADM non-Ig scaffold to a subject enhances the concentration of mature ADM (mature ADM-NH₂ according to SEQ ID No.: 4) in the circulation of a subject and thus, improves the status of subjects having dementia or being at risk of dementia.

Subject matter of the present invention is a method for:
a) diagnosing dementia, or
b) determining the risk of getting dementia in a subject that does not have dementia, or
c) monitoring therapy or monitoring or guiding intervention in a subject that has dementia, or
d) monitoring therapy or monitoring or guiding preventive intervention in a subject that is at risk of getting dementia,
   wherein the level of mature ADM-NH₂ according to SEQ ID No.: 4 is determined in a sample of bodily fluid of a subject and wherein said level of mature ADM-NH₂ is compared with a threshold level,
   and wherein
a) said subject is diagnosed with dementia if the level of mature ADM-NH₂ according to SEQ ID No.: 4 is below said threshold level, or wherein
b) said subject has an enhanced risk of getting dementia if said level of mature ADM-NH₂ according to SEQ ID No.: 4 is below said threshold level, or wherein
c) the status of a subject having dementia or being at risk of getting dementia is improving under therapy or intervention if said level of mature ADM-NH₂ according to SEQ ID No.: 4 is increased during the course of therapy or intervention and/or wherein intervention maybe continued if said level of mature ADM-NH₂ according to SEQ ID No.: 4 is increased above said level threshold.

In one embodiment subject matter of the present invention is a method for:
a) diagnosing dementia, or
b) determining the risk of getting dementia in a subject that does not have dementia, or
c) monitoring therapy or monitoring or guiding intervention in a subject that has dementia, or
d) monitoring preventive therapy or monitoring or guiding preventive intervention in a subject that is at risk of getting dementia,
   wherein a marker ratio is determined that maybe the ratio of the level of mature ADM-NH₂ according to SEQ ID No.: 4 determined in a sample of bodily fluid of said subject to the level of pro-Adrenomedullin or a fragment thereof (which is not mature ADM-NH₂ according to SEQ ID No.: 4) determined in a sample of bodily fluid of said subject and wherein said marker ratio is compared to a threshold ratio
   and wherein
a) said subject is diagnosed with dementia if the marker ratio ADM-NH₂/pro-Adrenomedullin or a fragment thereof is below said ratio threshold, or wherein
b) said subject has an enhanced risk of getting dementia if the marker ratio ADM-NH₂/pro-Adrenomedullin or a fragment thereof is below said ratio threshold, or wherein
c) the status of a subject having dementia or being at risk of getting dementia is improving under therapy or intervention if said marker ratio is increased during the course of therapy or intervention and wherein intervention maybe continued if said level of the marker ratio is increased above said ratio threshold.

Alternatively, the level of mature ADM-NH₂ according to SEQ ID No.: 4 determined in a sample of bodily fluid of said subject and the level of pro-Adrenomedullin or a fragment thereof (which is not mature ADM-NH₂ according to SEQ ID No.: 4) determined in a sample of bodily fluid of said subject will be combined in a mathematical formula or algorithm, wherein the result of said formula or algorithm is used for diagnosing dementia, or determining the risk of getting dementia in a subject that does not have dementia, or monitoring therapy or monitoring or guiding intervention in a subject that has dementia, or monitoring preventive therapy or monitoring or guiding preventive intervention in a subject that is at risk of getting dementia.

In any case in one embodiment of the invention the level of both markers is determined: the level of mature ADM-NH₂ according to SEQ ID No.: 4 determined in a sample of bodily fluid of said subject and the level of pro-Adrenomedullin or a fragment thereof (which is not mature ADM-NH₂ according to SEQ ID No.: 4) determined in a sample of bodily fluid of said subject. Both marker levels are used to conduct a calculation which maybe either a ratio, of both markers (e.g. ratio between mature ADM-NH₂ and pro-ADM or fragment thereof or ratio between proADM or fragment thereof and mature ADM-NH₂), or a mathematical formula in which both markers are introduced or a mathematical algorithm in which both markers are introduced. The outcome of such a ratio or mathematical formula or mathematical algorithm maybe a value that is then compared with a predetermined threshold value and this comparison is then used for diagnosing dementia, or determining the risk of getting dementia in a subject that does not have dementia, or monitoring therapy or monitoring or guiding intervention in a subject that has dementia, or monitoring preventive therapy or monitoring or guiding preventive intervention in a subject that is at risk of getting dementia.

In one embodiment of the subject matter of the present invention said fragment of pro-Adrenomedullin is selected from a group comprising PAMP (SEQ ID No. 2), MR-proADM (SEQ ID No. 3), ADM-Gly (SEQ ID No.: 5) and CT-proADM (SEQ ID No. 6).

In one embodiment of the subject matter of the present invention the threshold level of mature ADM-NH₂ according to SEQ ID No.: 4 is equal or below 15 pg/ml, preferably equal or below 10 pg/ml, preferably equal or below 5 pg/mL.

In one embodiment of the subject matter of the present invention the marker level ratio threshold is in a range of 0.2 to 0.75, preferably 0.3 to 0.6, preferably 0.4 to 0.5.

For the calculation of the ratio, the concentration of the two markers has to be preferably expressed in the same unit (e.g. pmol/L).

In one embodiment of the subject matter of the present invention the sample of bodily fluid is selected from the group of patients with mild cognitive impairment (MCI), Alzheimer' s disease, vascular dementia, mixed Alzheimer's disease and vascular dementia, Lewy body dementia, frontotemporal dementia, focal dementias (such as progressive aphasia), subcortical dementias (such as Parkinson's disease dementia, and secondary causes of dementia syndrome (such as intracranial lesions).

In one embodiment of the subject matter of the present invention the sample of bodily fluid is taken from a subject that has never had a diagnosis of dementia or MCI at the time of sample taking.

In one embodiment of the subject matter of the present invention at least one additional clinical parameter is determined selected from the group comprising age, race, mental status testing (e g mini-mental state examination (MMSE)), neuroimaging (CT, MRT, PET, SPECT), family history, ApoE4 genotype, Amyloidβ 1-42 (Aβ₁₋₄₂), Amyloidβ 1-40 (Aβ₁₋₄₀), total Tau-protein, phosphorylated Tau-protein (p-Tau 181, p-Tau 199, p-Tau 231).

In one embodiment of the subject matter of the present invention the level of said marker is determined by an immunoassay.

In one embodiment of the subject matter of the present invention said method is used for patient stratification to select a patient for treatment with an Anti-adrenomedullin (ADM) antibody or an anti-adrenomedullin antibody fragment or anti-ADM non-Ig scaffold for use in prevention and therapy of dementia in a subject, wherein said anti-ADM antibody or anti-ADM fragment or anti-ADM non-Ig scaffold binds to the N-terminal part (aa 1-21) of adrenomedullin:

(SEQ ID No. 21)
YRQSMNNFQGLRSFGCRFGTC.

Subject matter of the present invention is an Anti-adrenomedullin (ADM) antibody or an anti-adrenomedullin antibody fragment or anti-ADM non-Ig scaffold for use in prevention and therapy of dementia in a subject, wherein said anti-ADM antibody or anti-ADM fragment or anti-ADM non-Ig scaffold binds to the N-terminal part (aa 1-21) of Adrenomedullin:

(SEQ ID No. 21)
YRQSMNNFQGLRSFGCRFGTC.

Subject matter of the present invention is an Anti-adrenomedullin (ADM) antibody or an anti-adrenomedullin antibody fragment or anti-ADM non-Ig scaffold for use in prevention and therapy of dementia in a subject, wherein said subject has a level of mature ADM-NH₂ according to SEQ ID No.: 4 determined in a sample of bodily fluid of said subject below a threshold level and/or has a marker ratio that is the ratio of the level of mature ADM-NH₂ according to SEQ ID No.: 4 determined in a sample of bodily fluid of said subject to the level of pro-Adrenomedullin or a fragment thereof determined in a sample of bodily fluid of said subject and wherein said marker level ratio is below a ratio threshold.

Subject matter of the present invention is an Anti-adrenomedullin (ADM) antibody or an anti-adrenomedullin antibody fragment or anti-ADM non-Ig scaffold for use in prevention and therapy of dementia in a subject, wherein said fragment of pro-Adrenomedullin is selected from a group comprising PAMP (SEQ ID No. 2), MR-proADM (SEQ ID No. 3), ADM-Gly (SEQ ID No.: 5) and CT-proADM (SEQ ID No. 6).

Subject matter of the present invention is an Anti-adrenomedullin (ADM) antibody or an anti-adrenomedullin antibody fragment or anti-ADM non-Ig scaffold for use in prevention and therapy of dementia in a subject, wherein said subject is selected by a method as above explained.

Subject matter of the present invention is an Anti-adrenomedullin (ADM) antibody or an anti-adrenomedullin antibody fragment or anti-ADM non-Ig scaffold for use in prevention and therapy of dementia in a subject, wherein the threshold level of mature ADM-NH₂ according to SEQ ID No.: 4 is equal or below 15 pg/ml, preferably equal or below 10 pg/ml, preferably equal or below 5 pg/ml.

Subject matter of the present invention is an Anti-adrenomedullin (ADM) antibody or an anti-adrenomedullin antibody fragment or anti-ADM non-Ig scaffold for use in prevention and therapy of dementia in a subject, wherein the marker level ratio is in a range 0.2 to 0.75, preferably 0.3 to 0.6, preferably 0.4 to 0.5.

Subject matter of the present invention is an Anti-adrenomedullin (ADM) antibody or an anti-adrenomedullin antibody fragment or anti-ADM non-Ig scaffold for use in prevention and therapy of dementia in a subject, wherein said subject is selected according to a method as explained above, wherein the sample of bodily fluid is selected from the group of blood, serum, plasma, urine, cerebrospinal fluid (CSF), and saliva.

Subject matter of the present invention is an Anti-adrenomedullin (ADM) antibody or an anti-adrenomedullin antibody fragment or anti-ADM non-Ig scaffold for use in prevention and therapy of dementia in a subject, wherein at least one additional clinical parameter is determined selected from the group comprising age, race, mental status testing (e g mini-mental state examination (MMSE)), neuroimaging (CT, MRT, PET, SPECT), family history, ApoE4 genotype, Amyloidβ 1-42 (Aβ₁₋₄₂), Amyloidβ 1-40 (Aβ₁₋₄₀), total Tau-protein, phosphorylated Tau-protein (p-Tau 181, p-Tau 199, p-Tau 231).

Subject matter of the present invention is an Anti-adrenomedullin (ADM) antibody or an anti-adrenomedullin antibody fragment or anti-ADM non-Ig scaffold for use in prevention and therapy of dementia in a subject, wherein the level of said marker is determined by an immunoassay.

Mature ADM, bio-ADM and ADM -NH₂ is used synonymously throughout this application and is a molecule according to SEQ ID No.: 4.

As used herein, the term “PAMP” comprises both circulating forms of PAMP, namely a biologically inactive C-terminally Glycine-extended PAMP (PAMP-Gly) and a biologically active C-terminally amidated PAMP (PAMP-amide).

In a specific embodiment of the invention said proADM and/or fragments thereof having at least 5 amino acids and mature ADM is/are selected from the group comprising:

(pre-pro-Adrenomedullin (pre-proADM))-amino acids
1-185
SEQ ID No. 1
MKLVSVALMYLGSLAFLGADTARLDVASEFRKKWNKWALSRGKRELRMS
SSYPTGLADVKAGPAQTLIRPQDMKGASRSPEDSSPDAARIRVKRYRQS
MNNFQGLRSFGCRFGTCTVQKLAHQIYQFTDKDKDNVAPRSKISPQGYG
RRRRRSLPEAGPGRTLVSSKPQAHGAPAPPSGSAPHFL
(Proadrenomedullin N-20 terminal peptide, PAMP):
amino acids 22-41 of preproADM
SEQ ID No. 2
ARLDVASEF RKKWNKWALS R
(Midregional proAdrenomedullin, MR-proADM): amino
acids 45-92 of preproADM
SEQ ID No. 3
ELRMSS SYPTGLADVK AGPAQTLIRP QDMKGASRSP
EDSSPDAARI RV
(mature Adrenomedullin (mature ADM); amidated
ADM; bio-ADM; hADM): amino acids 95-146-CONH₂
SEQ ID No. 4
YRQSMN NFQGLRSFGC RFGTCTVQKL AHQIYQFTDK
DKDNVAPRSK ISPQGY-CONH₂
(Adrenomedullin 1-52-Gly (ADM 1-52-Gly)): amino
acids 95-147 of preproADM
SEQ ID No. 5
YRQSMN NFQGLRSFGC RFGTCTVQKL AHQIYQFTDK DKDNVAPRSK
ISPQGYG
(C-terminal proAdrenomedullin, CT-proADM): amino
acids 148-185 of preproADM
SEQ ID No. 6
RRR RRSLPEAGPG RTLVSSKPQA HGAPAPPSGS APHFL

In a specific embodiment of the invention the level of mature ADM-NH₂ (SEQ ID No. 4)—immunoreactivity in the bodily fluid of said subject is below a threshold.

In a specific embodiment of the invention the level of PAMP (SEQ ID No.: 2) immunoreactivity or the level of MR-proADM (SEQ ID No. 3) immunoreactivity or the level of CT-proADM (SEQ ID No. 6) immunoreactivity or the level of ADM 1-52-Gly (SEQ ID No. 5) immunoreactivity in the bodily fluid of said subject is above a threshold.

In a specific embodiment of the invention the ratio of the level of mature ADM-NH₂ (SEQ ID No.: 4) immunoreactivity and the level of MR-proADM (SEQ ID No. 3) immunoreactivity in the bodily fluid of said subject is below a threshold.

In a specific embodiment of the invention the level of mature ADM-NH₂ is determined by using at least one binder selected from the group: a binder that binds to a region comprised within the following sequence of mature ADM-NH₂ (SEQ ID No. 4) and a second binder that binds to a region comprised within the sequence of mature ADM-NH₂ (SEQ ID NO. 4).

In a specific embodiment of the invention the level of proADM and/or fragments thereof is determined by using at least one binder selected from the group: a binder that binds to a region comprised within the sequence of MR-proADM (SEQ ID No. 3) and a second binder that binds to a region comprised within the sequence of MR-proADM (SEQ ID No. 3).

In a specific embodiment of the invention the level of pro-ADM and/or fragments thereof is determined by using at least one binder selected from the group: a binder that binds to a region comprised within the sequence of CT-proADM (SEQ ID No. 6) and a second binder that binds to a region comprised within the sequence of CT-proADM (SEQ ID No. 6).

In a specific embodiment of the invention the level of pro-ADM and/or fragments thereof is determined by using at least one binder selected from the group: a binder that binds to a region comprised within the sequence of PAMP (SEQ ID No. 2) and a second binder that binds to a region comprised within the sequence of PAMP (SEQ ID No. 2).

In a specific embodiment of the invention the level of pro-ADM and/or fragments thereof is determined by using at least one binder selected from the group: a binder that binds to a region comprised within the sequence of ADM 1-52-Gly (SEQ ID No. 5) and a second binder that binds to a region comprised within the sequence of ADM 1-52-Gly (SEQ ID No. 5).

Subject matter of the present invention is a method according to the present invention, wherein the binder is selected from the group comprising an antibody, an antibody fragment or a non-Ig-Scaffold binding to Pro-Adrenomedullin or fragments thereof of at least 5 amino acids.

Another embodiment of the present application relates to a method according to the preceding embodiments, wherein said bodily fluid may be selected from the group comprising blood, serum, plasma, urine, cerebrospinal fluid (CSF), and saliva. In a more specific embodiment of the present invention said bodily fluid is a blood sample. A blood sample may be selected from the group comprising whole blood, serum and plasma. In a specific embodiment of the invention said sample is selected from the group comprising human citrate plasma, heparin plasma and EDTA plasma.

Subject matter of the present invention is a method according to the present invention, wherein said determination of Pro-Adrenomedullin or fragments thereof of at least 5 amino acids is performed more than once in one patient.

Subject matter of the present invention is a method according to the present invention, wherein said monitoring is performed in order to evaluate the response of said subject to preventive and/or therapeutic measures taken.

Subject matter of the present invention is a method according to the present invention, wherein said method is used in order to stratify said subjects into risk groups.

The term “risk”, as used herein, relates to the probability of suffering from an undesirable event or effect (e.g. a disease).

Another embodiment of the present application relates to a method according to the preceding embodiments, wherein a decrease of the level of mature ADM-NH₂ is predictive for an enhanced risk for getting a dementia.

Another embodiment of the present application relates to a method according to the preceding embodiments, wherein a decrease of the ratio between mature ADM-NH₂ and proADM or fragments thereof selected from the group comprising MR-proADM, CT-proADM, ADM-Gly and/or PAMP, is predictive for an enhanced risk of getting dementia.

Subject matter of the present invention is also a method for determining the risk of getting a dementia as defined in any of the preceding paragraphs, wherein said method is performed in order to stratify said subjects into risk groups as further defined below. In specific embodiments of the invention the methods are used in order to stratify the subjects into risk groups, e.g. those with a low risk, medium risk, or high risk to get a dementia disorder. Low risk of getting a dementia means that the value of mature ADM-NH₂ is substantially not decreased compared to a predetermined value in healthy subjects who did not get a dementia. A medium risk exists when the level of mature ADM-NH₂ is decreased compared to a predetermined value in healthy subjects who did not get a dementia disorder, and a high risk exists when the level of mature ADM-NH₂ is significantly decreased at baseline measurement and continues to decrease at subsequent analysis.

Risk of dementia means the risk of getting a dementia disorder within a certain period of time. In a specific embodiment said period of time is within 10 years, or within 7 years, or within 5 years or within 2.5 years.

The term “enhanced level” means a level above a certain threshold level.

The term “reduced level” means a level below a certain threshold level.

In a specific embodiment of the invention, an assay is used for determining the level of mature ADM-NH₂, wherein the assay sensitivity of said assay is <15 pg/ml, preferably <10 pg/ml, more preferred <5 pg/ml.

In a specific embodiment of the invention, an assay is used for determining the level MR-proADM, wherein the assay sensitivity of said assay is able to quantify MR-proADM of healthy subjects and is <0.5 nmol/L, preferably <0.4 nmol/L and more preferably <0.2 nmol/L.

In a specific embodiment of the invention, an assay is used for determining the level of CT-proADM, wherein the assay sensitivity of said assay is able to quantify CT-proADM of healthy subjects and is <100 pmol/L, preferably <75 pmol/L and more preferably <50 pmol/L.

Another embodiment of the present application relates to a method according to the preceding embodiments, wherein an assay is used for determining the level of PAMP-amide, wherein the assay sensitivity of said assay is able to quantify PAMP-amide of healthy subjects and is <0.3 pmol/L, preferably <0.2 pmol/L and more preferably <0.1 pmol/L.

Another embodiment of the present application relates to a method according to the preceding embodiments, wherein an assay is used for determining the level of PAMP-glycine, wherein the assay sensitivity of said assay is able to quantify PAMP-glycine of healthy subjects and is <0.5 pmol/L, preferably <0.25 pmol/L and more preferably <0.1 pmol/L.

Another embodiment of the present application relates to a method according to the preceding embodiments, wherein an assay is used for determining the level of ADM-Gly, wherein the assay sensitivity of said assay is able to quantify ADM-Gly of healthy subjects and is 60 pmol/L, preferably 10 pmol/L and more preferably 2 pmol/L.

In a specific embodiment of the invention, said binder exhibits a binding affinity to mature ADM-NH₂ or proADM and/or fragments thereof of at least 10⁷ M⁻¹, preferred 10⁸ M⁻¹, preferred affinity is greater than 10⁹ M⁻¹, most preferred greater than 10¹⁰ M⁻¹. A person skilled in the art knows that it may be considered to compensate lower affinity by applying a higher dose of compounds and this measure would not lead out-of-the-scope of the invention.

To determine the affinity of the antibodies to Adrenomedullin, the kinetics of binding of Adrenomedullin to immobilized antibody was determined by means of label-free surface plasmon resonance using a Biacore 2000 system (GE Healthcare Europe GmbH, Freiburg, Germany). Reversible immobilization of the antibodies was performed using an anti-mouse Fc antibody covalently coupled in high density to a CMS sensor surface according to the manufacturer's instructions (mouse antibody capture kit; GE Healthcare), (Lorenz et al. 2011. Antimicrob Agents Chemother. 55 (1): 165-173).

In a specific embodiment of the invention, said binder is selected from the group comprising an antibody or an antibody fragment or a non-Ig scaffold binding to mature ADM-NH₂ or proADM and/or fragments thereof.

In a specific embodiment of the invention, an assay is used for determining the level of mature ADM-NH₂ and/or proADM or fragments thereof having at least 5 amino acids, wherein such assay is a sandwich assay, preferably a fully automated assay.

In one embodiment of the invention it may be a so-called POC-test (point-of-care) that is a test technology, which allows performing the test within less than 1 hour near the patient without the requirement of a fully automated assay system. One example for this technology is the immunochromatographic test technology.

In one embodiment of the invention such an assay is a sandwich immunoassay using any kind of detection technology including but not restricted to enzyme label, chemiluminescence label, electrochemiluminescence label, preferably a fully automated assay. In one embodiment of the invention such an assay is an enzyme labeled sandwich assay. Examples of automated or fully automated assay comprise assays that may be used for one of the following systems: Roche Elecsys®, Abbott Architect®, Siemens Centauer®, Brahms Kryptor®, BiomerieuxVidas®, Alere Triage®, Ortho Clinical Diagnostics Vitros®.

A variety of immunoassays are known and may be used for the assays and methods of the present invention, these include: radioimmunoassays (“RIA”), homogeneous enzyme-multiplied immunoassays (“EMIT”), enzyme linked immunoadsorbent assays (“ELISA”), apoenzyme reactivation immunoassay (“ARIS”), dipstick immunoassays and immuno-chromatography assays.

In a specific embodiment of the invention, at least one of said two binders is labeled in order to be detected.

The preferred detection methods comprise immunoassays in various formats such as for instance radioimmunoassay (RIA), chemiluminescence- and fluorescence-immunoassays, Enzyme-linked immunoassays (ELISA), Luminex-based bead arrays, protein microarray assays, and rapid test formats such as for instance immunochromatographic strip tests.

In a preferred embodiment, said label is selected from the group comprising chemiluminescent label, enzyme label, fluorescence label, radioiodine label.

The assays can be homogenous or heterogeneous assays, competitive and non-competitive assays. In one embodiment, the assay is in the form of a sandwich assay, which is a non-competitive immunoassay, wherein the molecule to be detected and/or quantified is bound to a first antibody and to a second antibody. The first antibody may be bound to a solid phase, e.g. a bead, a surface of a well or other container, a chip or a strip, and the second antibody is an antibody which is labeled, e.g. with a dye, with a radioisotope, or a reactive or catalytically active moiety. The amount of labeled antibody bound to the analyte is then measured by an appropriate method. The general composition and procedures involved with “sandwich assays” are well-established and known to the skilled person (The Immunoassay Handbook, Ed. David Wild, Elsevier LTD, Oxford; 3rd ed. (May 2005); Hultschig et al. 2006. Curr Opin Chem Biol. 10 (1):4-10).

In another embodiment the assay comprises two capture molecules, preferably antibodies which are both present as dispersions in a liquid reaction mixture, wherein a first labelling component is attached to the first capture molecule, wherein said first labelling component is part of a labelling system based on fluorescence- or chemiluminescence-quenching or amplification, and a second labelling component of said marking system is attached to the second capture molecule, so that upon binding of both capture molecules to the analyte a measurable signal is generated that allows for the detection of the formed sandwich complexes in the solution comprising the sample.

In another embodiment, said labeling system comprises rare earth cryptates or rare earth chelates in combination with fluorescence dye or chemiluminescence dye, in particular a dye of the cyanine type.

In the context of the present invention, fluorescence based assays comprise the use of dyes, which may for instance be selected from the group comprising FAM (5- or 6-carboxyfluorescein), VIC, NED, Fluorescein, Fluoresceinisothiocyanate (FITC), IRD-700/800, Cyanine dyes, such as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethine dyes, and the like.

In the context of the present invention, chemiluminescence based assays comprise the use of dyes, based on the physical principles described for chemiluminescent materials in (Kirk-Othmer, Encyclopedia of chemical technology, 4th ed. 1993. John Wiley & Sons, Vol. 15: 518-562, incorporated herein by reference, including citations on pages 551-562). Preferred chemiluminescent dyes are acridinium esters.

As mentioned herein, an “assay” or “diagnostic assay” can be of any type applied in the field of diagnostics. Such an assay may be based on the binding of an analyte to be detected to one or more capture probes with a certain affinity. Concerning the interaction between capture molecules and target molecules or molecules of interest, the affinity constant is preferably greater than 10⁸ M⁻¹.

In the context of the present invention, “binder molecules” are molecules which may be used to bind target molecules or molecules of interest, i.e. analytes (i.e. in the context of the present invention ADM-NH₂ and/or proADM and fragments thereof), from a sample. Binder molecules have thus to be shaped adequately, both spatially and in terms of surface features, such as surface charge, hydrophobicity, hydrophilicity, presence or absence of lewis donors and/or acceptors, to specifically bind the target molecules or molecules of interest. Hereby, the binding may for instance be mediated by ionic, van-der-Waals, pi-pi, sigma-pi, hydrophobic or hydrogen bond interactions or a combination of two or more of the aforementioned interactions between the capture molecules and the target molecules or molecules of interest. In the context of the present invention, binder molecules may for instance be selected from the group comprising a nucleic acid molecule, a carbohydrate molecule, a PNA molecule, a protein, an antibody, a peptide or a glycoprotein. Preferably, the binder molecules are antibodies, including fragments thereof with sufficient affinity to a target or molecule of interest, and including recombinant antibodies or recombinant antibody fragments, as well as chemically and/or biochemically modified derivatives of said antibodies or fragments derived from the variant chain with a length of at least 12 amino acids thereof.

Chemiluminescent label may be acridinium ester label, steroid labels involving isoluminol labels and the like.

Enzyme labels may be lactate dehydrogenase (LDH), creatine kinase (CPK), alkaline phosphatase, aspartate aminotransferase (AST), alanine aminotransferase (ALT), acid phosphatase, glucose-6-phosphate dehydrogenase and so on.

In one embodiment of the invention at least one of said two binders is bound to a solid phase as magnetic particles, and polystyrene surfaces.

In a specific embodiment of the invention at least one of said two binders is bound to a solid phase.

In a specific embodiment of the invention the threshold of the ratio of the level of mature ADM-NH₂ according to SEQ ID No.: 4 determined in a sample of bodily fluid of said subject to the level of pro-Adrenomedullin or a fragment thereof (which is not mature ADM-NH₂ according to SEQ ID No.: 4) is within a range that is between 0.2 to 0.75, preferably 0.3 to 0.6, preferably 0.4 to 0.5 is applied.

The ADM-NH₂ levels of the present invention or proADM levels or fragments thereof, respectively, have been determined with the described ADM-NH₂ assay, as outlined in the examples (or proADM or fragments thereof assays, respectively). The mentioned threshold values above might be different in other assays, if these have been calibrated differently from the assay systems used in the present invention. Therefore, the mentioned cut-off values above shall apply for such differently calibrated assays accordingly, taking into account the differences in calibration. One possibility of quantifying the difference in calibration is a method comparison analysis (correlation) of the assay in question with the respective biomarker assay used in the present invention by measuring the respective biomarker (e.g. bio-ADM) in samples using both methods. Another possibility is to determine with the assay in question, given this test has sufficient analytical sensitivity, the median biomarker level of a representative normal population, compare results with the median biomarker levels as described in the literature and recalculate the calibration based on the difference obtained by this comparison. With the calibration used in the present invention, samples from normal (healthy) subjects have been measured: median plasma bio-ADM (mature ADM-NH₂) was 13.7 pg/ml (inter quartile range [IQR] 9.6-18.7 pg/mL) (Weber et al. 2017. JALM, 2(2): 222-233).

The plasma median MR-proADM concentration in normal (healthy) subjects was 0.41 (interquartile range 0.23-0.64) nmol/L (Smith et al. 2009. Clin Chem 55:1593-1595) using the automated sandwich fluorescence assay for the detection of MR-proADM as described in Caruhel et al. (Caruhel et al. 2009. Clin Biochem 42:725-8).

The plasma median concentration of CT-proADM in normal healthy subjects (n=200) was 77.6 pmol/L (min 46.6 pmol/L, max 136.2 pmol/L) and the 95% percentile was 113.8 pmol/L (EP 2 111 552 B1).

The plasma concentration of PAMP-amide in normal healthy subjects (n=51) was 0.51±0.19 pmol/L (mean±SD) (Hashida et al. 2004. Clin Biochem 37: 14-21).

The plasma concentration of PAMP-glycine in normal healthy subjects (n=51) was 1.15±0.38 pmol/L (mean±SD) (Hashida et al. 2004. Clin Biochem 37: 14-21).

In one embodiment the threshold may be pre-determined as follows:
Comparison of concentration of the marker in a bodily fluid obtained from said subject with the median of the marker in a bodily fluid obtained from an ensemble of pre-determined samples in a randomly selected population of subjects having comparable baseline conditions as said subject,
Comparison of concentration of the marker in a bodily fluid obtained from said subject with a quantile of the levels of the marker, and/or its fragments in a bodily fluid obtained from an ensemble of pre-determined samples in a population of subjects having comparable baseline conditions as said subject,
Calculation based on Cox Proportional Hazards analysis or by using Risk index calculations such as the NRI (Net Reclassification Index) or the IDI (Integrated Discrimination Index).

In addition, at least one clinical parameter or biomarker may be determined selected from the group comprising: age, race, mental status testing (e.g. mini-mental state examination (MMSE)), neuroimaging (CT, MRT, PET, SPECT), family history, ApoE4 genotype, Amyloidβ 1-42 (Aβ₁₋₄₂), Amyloidβ 1-40 (Aβ₁₋₄₀), total Tau-protein, phosphorylated Tau-protein (p-Tau 181, p-Tau 199, p-Tau 231).

In the context of the present invention the term “dementia” includes Alzheimer's disease, vascular dementia, mixed Alzheimer's disease and vascular dementia, Lewy body dementia frontotemporal dementia, focal dementias (such as progressive aphasia), subcortical dementias (such as Parkinson's disease dementia), and secondary causes of dementia syndrome (such as intracranial lesions).

In a more specific embodiment of the invention said dementia is selected from the group of Alzheimer's disease, vascular dementia and mixed Alzheimer's disease and vascular dementia.

Most preferred said dementia is Alzheimer's disease.

Another subject of the present invention is a pharmaceutical composition comprising the herein disclosed binder of the invention, specifically comprising an anti-ADM antibody or an anti-ADM antibody fragment or an anti-ADM non-Ig scaffold for use in the prevention or treatment of dementia.

In another embodiment of the present invention said pharmaceutical composition is a solution, preferably a ready-to-use solution.

In another embodiment of the present invention said pharmaceutical composition is a solution, preferably a ready-to-use solution comprising PBS at a pH of 7.4.

In another embodiment of the present invention said pharmaceutical composition is in a dried state that is to be reconstituted before use.

In another embodiment of the present invention said pharmaceutical composition is in a freeze-dried state that is to be reconstituted before use.

In another embodiment of the present invention said pharmaceutical composition that is to be used in the prevention and/or treatment of dementia is administered orally, epicutaneously, subcutaneously, intradermally, sublingually, intramuscularly, intraarterially, intracerebrally, intracerebroventricularly, intrathecally, intravenously, or via intraperitoneal administration. In a preferred embodiment of the present invention said pharmaceutical composition is administered intravenously. In another preferred embodiment of the present invention said pharmaceutical composition is administered via the central nervous system (CNS), e.g. intracerebrally, intracerebroventricularly and intrathecally.

An antibody according to the present invention is a protein including one or more polypeptides substantially encoded by immunoglobulin genes that specifically binds an antigen. The recognized immunoglobulin genes include the kappa, lambda, alpha (IgA), gamma (IgGi, IgG₂, IgG₃, IgG₄), delta (IgD), epsilon (IgE) and mu (IgM) constant region genes, as well as the myriad immunoglobulin variable region genes. Full-length immunoglobulin light chains are generally about 25 Kd or 214 amino acids in length. Full-length immunoglobulin heavy chains are generally about 50 Kd or 446 amino acid in length. Light chains are encoded by a variable region gene at the NH₂-terminus (about 110 amino acids in length) and a kappa or lambda constant region gene at the COOH-terminus. Heavy chains are similarly encoded by a variable region gene (about 116 amino acids in length) and one of the other constant region genes.

The basic structural unit of an antibody is generally a tetramer that consists of two identical pairs of immunoglobulin chains, each pair having one light and one heavy chain. In each pair, the light and heavy chain variable regions bind to an antigen, and the constant regions mediate effector functions. Immunoglobulins also exist in a variety of other forms including, for example, Fv, Fab, and (Fab′)₂, as well as bifunctional hybrid antibodies and single chains (e.g., Lanzavecchia et al. 1987. Eur. J. Immunol. 17:105; Huston et al. 1988, Proc. Natl. Acad. Sci. U.S.A., 85:5879-5883; Bird et al. 1988, Science 242:423-426; Hood et al.,Immunology, Benjamin, N.Y., 2nd ed., 1984; Hunkapiller and Hood 1986. Nature 323:15-16). An immunoglobulin light or heavy chain variable region includes a framework region interrupted by three hypervariable regions, also called complementarity determining regions (CDR's) (see, Sequences of Proteins of Immunological Interest, E. Kabat et al., U.S. Department of Health and Human Services, 1983). As noted above, the CDRs are primarily responsible for binding to an epitope of an antigen. An immune complex is an antibody, such as a monoclonal antibody, chimeric antibody, humanized antibody or human antibody, or functional antibody fragment, specifically bound to the antigen.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody can be joined to human constant segments, such as kappa and gamma 1 or gamma 3. In one example, a therapeutic chimeric antibody is thus a hybrid protein composed of the variable or antigen-binding domain from a mouse antibody and the constant or effector domain from a human antibody, although other mammalian species can be used, or the variable region can be produced by molecular techniques. Methods of making chimeric antibodies are well known in the art (e.g., see U.S. Pat. No. 5,807,715). A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions, which have substantially no effect on antigen binding or other immunoglobulin functions. Exemplary conservative substitutions are those such as gly, ala; val, ile, leu; asp, glu; asn, gln; ser, thr; lys, arg; and phe, tyr. Humanized immunoglobulins can be constructed by means of genetic engineering (e.g., see U.S. Pat. No. 5,585,089). A human antibody is an antibody wherein the light and heavy chain genes are of human origin. Human antibodies can be generated using methods known in the art. Human antibodies can be produced by immortalizing a human B cell secreting the antibody of interest Immortalization can be accomplished, for example, by EBV infection or by fusing a human B cell with a myeloma or hybridoma cell to produce a trioma cell. Human antibodies can also be produced by phage display methods (see, e.g., Dower et al., PCT Publication No. WO91/17271; McCafferty et al., PCT Publication No. WO92/001047; and Winter, PCT Publication No. WO92/20791), or selected from a human combinatorial monoclonal antibody library (see the Morphosys website). Human antibodies can also be prepared by using transgenic animals carrying a human immunoglobulin gene (for example, see Lonberget al., PCT Publication No. WO93/12227; and Kucherlapati, PCT Publication No. WO91/10741).

Thus, the antibody may have the formats known in the art. Examples are human antibodies, monoclonal antibodies, humanized antibodies, chimeric antibodies, CDR-grafted antibodies. In a preferred embodiment antibodies according to the present invention are recombinantly produced antibodies as e.g. IgG, a typical full-length immunoglobulin, or antibody fragments containing at least the F-variable domain of heavy and/or light chain as e.g. chemically coupled antibodies (fragment antigen binding) including but not limited to Fab-fragments including Fab minibodies, single chain Fab antibody, monovalent Fab antibody with epitope tags, e.g. Fab-V5Sx2; bivalent Fab (mini-antibody) dimerized with the CH3 domain; bivalent Fab or multivalent Fab, e.g. formed via multimerization with the aid of a heterologous domain, e.g.

via dimerization of dHLXdomains, e.g. Fab-dHLX-FSx2; F(ab′)2-fragments, scFv-fragments, multimerized multivalent or/and multispecific scFv-fragments, bivalent and/or bispecific diabodies, BITE® (bispecific T-cell engager), trifunctional antibodies, polyvalent antibodies, e.g. from a different class than G; single-domain antibodies, e.g. nanobodies derived from camelid or fish immunoglobulines and numerous others.

In addition to antibodies other biopolymer scaffolds are well known in the art to complex a target molecule and have been used for the generation of highly target specific biopolymers. Examples are aptamers, spiegelmers, anticalins and conotoxins.

In a preferred embodiment the antibody format is selected from the group comprising Fv fragment, scFv fragment, Fab fragment, scFab fragment, (Fab)2 fragment and scFv-Fc Fusion protein. In another preferred embodiment the antibody format is selected from the group comprising scFab fragment, Fab fragment, scFv fragment and bioavailability optimized conjugates thereof, such as PEGylated fragments. One of the most preferred formats is the scFab format.

Non-Ig scaffolds may be protein scaffolds and may be used as antibody mimics as they are capable to bind to ligands or antigenes. Non-Ig scaffolds may be selected from the group comprising tetranectin-based non-Ig scaffolds (e.g. described in US 2010/0028995), fibronectin scaffolds (e.g. described in EP 1 266 025), lipocalin-based scaffolds (e.g. described in WO 2011/154420); ubiquitin scaffolds (e.g. described in WO 2011/073214), transferring scaffolds (e.g. described in US 2004/0023334), protein A scaffolds (e.g. described in EP 2 231 860), ankyrin repeat based scaffolds (e.g. described in WO 2010/060748), microprotein scaffolds (preferably microproteins forming a cystine knot) (e.g. described in EP 2 314 308), Fyn SH3 domain based scaffolds (e.g. described in WO 2011/023685) EGFR-A-domain based scaffolds (e.g. described in WO 2005/040229) and Kunitz domain based scaffolds (e.g. described in EP 1941867).

Furthermore, in one embodiment of the invention an anti-Adrenomedullin (ADM) antibody or an anti-adrenomedullin antibody fragment or an anti-ADM non-Ig scaffold is monospecific.

Monospecific anti-adrenomedullin (ADM) antibody or monospecific anti-adrenomedullin antibody fragment or monospecific anti-ADM non-Ig scaffold means that said antibody or antibody fragment or non-Ig scaffold binds to one specific region encompassing at least 5 amino acids within the target ADM. Monospecific anti-Adrenomedullin (ADM) antibody or monospecific anti-adrenomedullin antibody fragment or monospecific anti-ADM non-Ig scaffold are anti-adrenomedullin (ADM) antibodies or anti-adrenomedullin antibody fragments or anti-ADM non-Ig scaffolds that all have affinity for the same antigen.

In another specific and preferred embodiment the anti-ADM antibody or the anti-ADM antibody fragment or anti-ADM non-Ig scaffold binding to ADM is a monospecific antibody, antibody fragment or non-Ig scaffold, respectively, whereby monospecific means that said antibody or antibody fragment or non-Ig scaffold binds to one specific region encompassing at least 4 amino acids within the target ADM. Monospecific antibodies or fragments or non-Ig scaffolds according to the invention are antibodies or fragments or non-Ig scaffolds that all have affinity for the same antigen. Monoclonal antibodies are monospecific, but monospecific antibodies may also be produced by other means than producing them from a common germ cell.

Said anti-ADM antibody or antibody fragment binding to ADM or non-Ig scaffold binding to ADM may be a non-neutralizing anti-ADM antibody or antibody fragment binding to ADM or non-Ig scaffold binding to ADM.

In a specific embodiment said anti-ADM antibody, anti-ADM antibody fragment or anti-ADM non-Ig scaffold is a non-neutralizing antibody, fragment or non-Ig scaffold. A neutralizing anti-ADM antibody, anti-ADM antibody fragment or anti-ADM non-Ig scaffold would block the bioactivity of ADM to nearly 100%, to at least more than 90%, preferably to at least more than 95%.

In contrast, a non-neutralizing anti-ADM antibody, or anti-ADM antibody fragment or anti-ADM non-Ig scaffold blocks the bioactivity of ADM less than 100%, preferably to less than 95%, preferably to less than 90%, more preferred to less than 80% and even more preferred to less than 50%. This means that bioactivity of ADM is reduced to less than 100%, to 95% or less but not more, to 90% or less but not more , to 80% or less but not more , to 50% or less but not more This means that the residual bioactivity of ADM bound to the non-neutralizing anti-ADM antibody, or anti-ADM antibody fragment or anti-ADM non-Ig scaffold would be more than 0%, preferably more than 5%, preferably more than 10% , more preferred more than 20%, more preferred more than 50%.

In this context (a) molecule(s), being it an antibody, or an antibody fragment or a non-Ig scaffold with “non-neutralizing anti-ADM activity”, collectively termed here for simplicity as “non-neutralizing” anti-ADM antibody, antibody fragment, or non-Ig scaffold, that e.g. blocks the bioactivity of ADM to less than 80%, is defined as
a molecule or molecules binding to ADM, which upon addition to a culture of an eukaryotic cell line, which expresses functional human recombinant ADM receptor composed of CRLR (calcitonin receptor like receptor) and RAMP3 (receptor-activity modifying protein 3), reduces the amount of cAMP produced by the cell line through the action of parallel added human synthetic ADM peptide, wherein said added human synthetic ADM is added in an amount that in the absence of the non-neutralizing antibody to be analyzed, leads to half-maximal stimulation of cAMP synthesis, wherein the reduction of cAMP by said molecule(s) binding to ADM takes place to an extent, which is not more than 80%, even when the non-neutralizing molecule(s) binding to ADM to be analyzed is added in an amount, which is 10-fold more than the amount, which is needed to obtain the maximal reduction of cAMP synthesis obtainable with the non-neutralizing antibody to be analyzed.

The same definition applies to the other ranges; 95%, 90%, 50% etc.

In a preferred embodiment of the present invention said anti-ADM antibody or an anti-adrenomedullin antibody fragment or anti-ADM non-Ig scaffold binds to a region or epitope of ADM that is located in the N-terminal part (aa 1-21) of adrenomedullin.

In another preferred embodiment said anti-ADM-antibody or an anti-adrenomedullin antibody fragment or anti-ADM non-Ig scaffold recognizes and binds to a region or epitope within amino acids 1-14 (SEQ ID No.: 27) of adrenomedullin; that means to the N-terminal part (aa 1-14) of adrenomedullin. In another preferred embodiment said anti-ADM-antibody or an anti-adrenomedullin antibody fragment or anti-ADM non-Ig scaffold recognizes and binds to a region or epitope within amino acids 1-10 of adrenomedullin (SEQ ID No.: 28); that means to the N-terminal part (aa 1-10) of adrenomedullin.

aa 1-14 of ADM
(SEQ ID No.: 27)
YRQSMNNFQGLRSF
aa 1-10 of ADM
(SEQ ID No.: 28)
YRQSMNNFQG

In another preferred embodiment said anti-ADM antibody or an anti-adrenomedullin antibody fragment or anti-ADM non-Ig scaffold recognizes and binds to a region or epitope within amino acids 1-6 of adrenomedullin (SEQ ID No.: 29); that means to the N-terminal part (aa 1-6) of adrenomedullin. As above stated said region or epitope comprises preferably at least 4 or at least 5 amino acids in length.

aa 1-6 of ADM
(SEQ ID No.: 29)
YRQSMN

In another preferred embodiment said anti-ADM antibody or an anti-adrenomedullin antibody fragment or anti-ADM non-Ig scaffold recognizes and binds to the N-terminal end (aa1) of adrenomedullin. N-terminal end means that the amino acid 1, that is “Y” of SEQ ID No. 4, 5, 21, 27, 28 or 29; is mandatory for antibody binding. The antibody or fragment or scaffold would neither bind N-terminal extended nor N-terminal modified Adrenomedullin nor N-terminal degraded adrenomedullin. This means in another preferred embodiment said anti-ADM-antibody or an anti-adrenomedullin antibody fragment or anti-ADM non-Ig scaffold binds only to a region within the sequence of mature ADM if the N-terminal end of ADM is free. In said embodiment the anti-ADM antibody or anti-adrenomedullin antibody fragment or non-Ig scaffold would not bind to a region within the sequence of mature ADM if said sequence is e.g. comprised within pro-ADM.

For the sake of clarity the numbers in brackets for specific regions of ADM like “N-terminal part (aa 1-21)” is understood by a person skilled in the art that the N-terminal part of ADM consists of amino acids 1-21 of the mature ADM sequence.

In one embodiment of the invention antibodies according to the present invention may be produced as follows:

A Balb/c mouse was immunized with ADM-100 μg Peptide-BSA-Conjugate at day 0 and 14 (emulsified in 100 μl complete Freund's adjuvant) and 50 μg at day 21 and 28 (in 100 μl incomplete Freund's adjuvant). Three days before the fusion experiment was performed, the animal received 50 μg of the conjugate dissolved in 100 μl saline, given as one intraperitoneal and one intravenous injection.

Splenocytes from the immunized mouse and cells of the myeloma cell line SP2/0 were fused with lml 50% polyethylene glycol for 30 s at 37° C. After washing, the cells were seeded in 96-well cell culture plates. Hybrid clones were selected by growing in HAT medium [RPMI 1640 culture medium supplemented with 20% fetal calf serum and HAT-Supplement]. After two weeks the HAT medium is replaced with HT Medium for three passages followed by returning to the normal cell culture medium.

The cell culture supernatants were primary screened for antigen specific IgG antibodies three weeks after fusion. The positive tested microcultures were transferred into 24-well plates for propagation. After retesting, the selected cultures were cloned and recloned using the limiting-dilution technique and the isotypes were determined (see also Lane 1985. J. Immunol. Meth. 81: 223-228; Ziegler, B. et al. 1996 Horm. Metab. Res. 28: 11-15).

Antibodies may be produced by means of phage display according to the following procedure:

The human naive antibody gene libraries HAL7/8 were used for the isolation of recombinant single chain F-Variable domains (scFv) against adrenomedullin peptide. The antibody gene libraries were screened with a panning strategy comprising the use of peptides containing a biotin tag linked via two different spacers to the adrenomedullin peptide sequence. A mix of panning rounds using non-specifically bound antigen and streptavidin bound antigen were used to minimize background of non-specific binders. The eluted phages from the third round of panning have been used for the generation of monoclonal scFv expressing E. coli strains. Supernatant from the cultivation of these clonal strains has been directly used for an antigen ELISA testing (see Hust et al. 2011. Journal of Biotechnology 152: 159-170; Schütte et al. 2009. PLoS One 4, e6625).

Humanization of murine antibodies may be conducted according to the following procedure:

For humanization of an antibody of murine origin the antibody sequence is analyzed for the structural interaction of framework regions (FR) with the complementary determining regions (CDR) and the antigen. Based on structural modelling an appropriate FR of human origin is selected and the murine CDR sequences are transplanted into the human FR. Variations in the amino acid sequence of the CDRs or FRs may be introduced to regain structural interactions, which were abolished by the species switch for the FR sequences. This recovery of structural interactions may be achieved by random approach using phage display libraries or via directed approach guided by molecular modelling (Almagro et al. 2008. Front Biosci. 2008; 13:1619-33).

Another embodiment of the present application relates to a method according to the preceding embodiments, wherein the anti-ADM antibody for the treatment of the subject which binds to the N-terminal part, aa 1-21, of adrenomedullin, is a human CDR-grafted antibody or antibody fragment thereof that binds to ADM, wherein the human CDR-grafted antibody or antibody fragment thereof comprises an antibody heavy chain (H chain) comprising:

SEQ ID NO.7:
GYTFSRYW
SEQ ID NO. 8:
ILPGSGST
and/or
SEQ ID NO. 9:
TEGYEYDGFDY

and/or further comprises an antibody light chain (L chain) comprising:

SEQ ID NO. 10:
QSIVYSNGNTY
SEQ ID NO. 11: (Not mentioned in the sequence
listing due to the length of 3 amino acids.)
RVS
and/or
SEQ ID NO. 12:
FQGSHIPYT.

In another specific embodiment of the present application the anti-ADM antibody for the treatment of the subject is a human monoclonal antibody that binds to ADM or an antibody fragment thereof wherein the heavy chain comprises at least one CDR selected from the group comprising:

SEQ ID NO. 7:
GYTFSRYW
SEQ ID NO. 8:
ILPGSGST
SEQ ID NO. 9:
TEGYEYDGFDY

and wherein the light chain comprises at least one CDR selected from the group comprising:

SEQ ID NO. 10:
QSIVYSNGNTY
SEQ ID NO. 11: (Not mentioned in the sequence
listing due to the length of 3 amino acids.)
RVS
SEQ ID NO. 12:
FQGSHIPYT.

In another embodiment of the present application, the anti-ADM antibody for the treatment of the subject is a human monoclonal antibody that binds to ADM or an antibody fragment thereof wherein the heavy chain comprises the sequences

SEQ ID NO. 7:
GYTFSRYW
SEQ ID NO. 8:
ILPGSGST
SEQ ID NO. 9:
TEGYEYDGFDY

and wherein the light chain comprises the sequences

SEQ ID NO. 10:
QSIVYSNGNTY
SEQ ID NO. 11: (Not mentioned in the sequence
listing due to the length of 3 amino acids.)
RVS
SEQ ID NO. 12:
FQGSHIPYT.

Another embodiment of the present application relates to a method of the preceding embodiment, wherein said antibody or fragment for the treatment is a human monoclonal antibody or fragment that binds to ADM or an antibody fragment thereof wherein the heavy chain comprises the sequences

CDR1:
SEQ ID NO. 7:
GYTFSRYW
CDR2:
SEQ ID NO. 8:
ILPGSGST
CDR3:
SEQ ID NO. 9:
TEGYEYDGFDY

and wherein the light chain comprises the sequences

CDR1:
SEQ ID NO. 10:
QSIVYSNGNTY
CDR2:
SEQ ID NO. 11: (Not mentioned in the sequence
listing due to the length of 3 amino acids.)
RVS
CDR3:
SEQ ID NO. 12:
FQGSHIPYT.

Another embodiment of the present application relates to a method of the preceding embodiment, wherein said antibody or fragment for the treatment comprises the following sequences as a VH region:

SEQ ID NO. 13 (AM-VH-C):
QVQLQQSGAELMKPGASVKISCKATGYTFSRYWIEWVKQRPGHGLEWIGE
ILPGSGSTNYNEKFKGKATITADTSSNTAYMQLSSLTSEDSAVYYCTEGY
EYDGFDYWGQGTTLTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKHHHHHH
SEQ ID NO. 14 (AM-VH1):
QVQLVQSGAEVKKPGSSVKVSCKASGYTFSRYWISWVRQAPGQGLEWMGR
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKHHHHHH
SEQ ID NO. 15 (AM-VH2-E40):
QVQLVQSGAEVKKPGSSVKVSCKASGYTFSRYWIEWVRQAPGQGLEWMGR
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKHHHHHH
SEQ ID NO. 16 (AM-VH3-T26-E55):
QVQLVQSGAEVKKPGSSVKVSCKATGYTFSRYWISWVRQAPGQGLEWMGE
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKHHHHHH;
or
SEQ ID NO. 17 (AM-VH4-T26-E40-E55):
QVQLVQSGAEVKKPGSSVKVSCKATGYTFSRYWIEWVRQAPGQGLEWMGE
ILPGSGSTNYAQKFQGRVTITADESTSTAYMELSSLRSEDTAVYYCTEGY
EYDGFDYVVGQGTTVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKHHHHHH;

and comprises the following sequence as a VL region:

SEQ ID NO. 18 (AM-VL-C):
DVLLSQTPLSLPVSLGDQATISCRSSQSIVYSNGNTYLEWYLQKPGQSPK
LLIYRVSNRFSGVPDRFSGSGSGTDFTLKISRVEAEDLGVYYCFQGSHIP
YTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
SEQ ID NO. 19 (AM-VL1):
DVVMTQSPLSLPVTLGQPASISCRSSQSIVYSNGNTYLNWFQQRPGQSPR
RLIYRVSNRDSGVPDRFSGSGSGTDFTLKISRVEAEDVGVYYCFQGSHIP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
SEQ ID NO. 20 (AM-VL2-E40):
DVVMTQSPLSLPVTLGQPASISCRSSQSIVYSNGNTYLEWFQQRPGQSPR
RLIYRVSNRDSGVPDRFSGSGSGTDFTLKISRVEAEDVGVYYCFQGSHIP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC.

The following embodiments are subject of the present invention:

1) A method for:
a) diagnosing dementia, or
b) determining the risk of getting dementia in a subject that does not have dementia, or
c) monitoring therapy or monitoring or guiding intervention in a subject that has dementia, or
d) monitoring therapy or monitoring or guiding preventive intervention in a subject that is at risk of getting dementia,
   wherein the level of mature ADM-NH₂ according to SEQ ID No.: 4 is determined in a sample of bodily fluid of a subject and wherein said level of mature ADM-NH₂ is compared with a threshold level,
   and wherein
a) said subject is diagnosed with dementia if the level of mature ADM-NH₂ according to SEQ ID No.: 4 is below said threshold level, or wherein
b) said subject has an enhanced risk of getting dementia if said level of mature ADM-NH₂ according to SEQ ID No.: 4 is below said threshold level, or wherein
c) the status of a subject having dementia or being at risk of getting dementia is improving under therapy or intervention if said level of mature ADM-NH₂ according to SEQ ID No.: 4 is increased during the course of therapy or intervention and/or wherein intervention maybe continued if said level of mature ADM-NH₂ according to SEQ ID No.: 4 is increased above said threshold.

2) A method for:
a) diagnosing dementia, or
b) determining the risk of getting dementia in a subject that does not have dementia, or
c) monitoring therapy or monitoring or guiding intervention in a subject that has dementia, or
d) monitoring preventive therapy or monitoring or guiding preventive intervention in a subject that is at risk of getting dementia,
   wherein a marker ratio is determined that maybe ratio of the level of mature ADM-NH₂ according to SEQ ID No.: 4 determined in a sample of bodily fluid of said subject to the level of pro-Adrenomedullin or a fragment thereof (which is not mature ADM-NH₂ according to SEQ ID No.: 4) determined in a sample of bodily fluid of said subject and wherein said marker ratio is compared to a threshold, ratio
   and wherein
a) said subject is diagnosed with dementia if the marker ratio mature ADM-NH_{2/}pro-Adrenomedullin or a fragment thereof is below said ratio threshold, or wherein
b) said subject has an enhanced risk of getting dementia if the marker ratio of mature ADM-NH₂/pro-Adrenomedullin or a fragment thereof is below said ratio threshold, or wherein
c) the status of a subject having dementia or being at risk of getting dementia is improving under therapy or intervention if said marker ratio is increased during the course of therapy or intervention
   or alternatively to the above marker ratio the level of mature ADM-NH₂ according to SEQ ID No.: 4 is determined in a sample of bodily fluid of said subject and the level of pro-Adrenomedullin or a fragment thereof (which is not mature ADM-NH₂ according to SEQ ID No.: 4) is determined in a sample of bodily fluid of said subject and both determined level are combined in a mathematical algorithm, wherein the result of said mathematical algorithm is used for diagnosing dementia, or determining the risk of getting dementia in a subject that does not have dementia, or monitoring therapy or monitoring or guiding intervention in a subject that has dementia, or monitoring preventive therapy or monitoring or guiding preventive intervention in a subject that is at risk of getting dementia.

3. A method according to item 2, wherein said fragment of pro-Adrenomedullin is selected from a group comprising PAMP (SEQ ID No. 2), MR-proADM (SEQ ID No. 3), ADM-Gly (SEQ ID No.: 5) and CT-proADM (SEQ ID No. 6).

4. A method according to item 1, wherein the threshold level of mature ADM-NH₂ according to SEQ ID No.: 4 is is equal or below 15 pg/ml, preferably equal or below 10 pg/ml, preferably equal or below 5 pg/ml.

5. A method according to item 2 or 3, wherein the ratio threshold is in a range of 0.2 to 0.75, preferably 0.3 to 0.6, preferably 0.4 to 0.5.

6. A method according to any of the preceding items, wherein said sample is selected from the group of blood, serum, plasma, urine, cerebrospinal fluid (CSF), and saliva.

7. A method according to items 1 to 6, wherein the sample of bodily fluid is taken from a subject that has never had a diagnosis of dementia or MCI at the time of sample taking.

8. A method according to any of the preceding items, wherein at least one additional clinical parameter is determined selected from the group comprising age, race, mental status testing (e g mini-mental state examination (MMSE)), neuroimaging (CT, MRT, PET, SPECT), family history, ApoE4 genotype, Amyloidβ 1-42 (Aβ₁₋₄₂), Amyloidβ 1-40 (Aβ₁₋₄₀), total Tau-protein, phosphorylated Tau-protein (p-Tau 181, p-Tau 199, p-Tau 231).

9. A method according to any of the preceding items, wherein the level of said marker is determined by an immunoassay.

10. A method according to any of the preceding items, wherein said method is used for patient stratification to select a patient for treatment with an Anti-adrenomedullin (ADM) antibody or an anti-adrenomedullin antibody fragment or anti-ADM non-Ig scaffold for use in prevention and therapy of dementia in a subject, wherein said anti-ADM antibody or anti-ADM fragment or anti-ADM non-Ig scaffold binds to the N-terminal part (aa 1-21) of adrenomedullin:

(SEQ ID No. 21)
YRQSMNNFQGLRSFGCRFGTC.

11. Anti-adrenomedullin (ADM) antibody or an anti-adrenomedullin antibody fragment or anti-ADM non-Ig scaffold for use in prevention and therapy of dementia in a subject, wherein said anti-ADM antibody or anti-ADM fragment or anti-ADM non-Ig scaffold binds to the N-terminal part (aa 1-21) of adrenomedullin:

(SEQ ID No. 21)
YRQSMNNFQGLRSFGCRFGTC.

12. Anti-adrenomedullin (ADM) antibody or an anti-adrenomedullin antibody fragment or anti-ADM non-Ig scaffold for use in prevention and therapy of dementia in a subject according to item 11, wherein said subject has a level of mature ADM-NH₂ according to SEQ ID No.: 4 determined in a sample of bodily fluid of said subject below a threshold level and/or has a marker ratio that is the ratio of the level of mature ADM-NH₂ according to SEQ ID No.: 4 determined in a sample of bodily fluid of said subject to the level of pro-Adrenomedullin or a fragment thereof determined in a sample of bodily fluid of said subject and wherein said marker level ratio is below a ratio threshold,

13. Anti-adrenomedullin (ADM) antibody or an anti-adrenomedullin antibody fragment or anti-ADM non-Ig scaffold for use in prevention and therapy of dementia in a subject according to item 12 wherein, said fragment of pro-Adrenomedullin is selected from a group comprising PAMP (SEQ ID No. 2), MR-proADM (SEQ ID No. 3), ADM-Gly (SEQ ID No.: 5) and CT-proADM (SEQ ID No. 6).

14. Anti-adrenomedullin (ADM) antibody or an anti-adrenomedullin antibody fragment or anti-ADM non-Ig scaffold for use in prevention and therapy of dementia in a subject according to items 11 to 13, wherein said subject is selected by a method according item 10.

15. Anti-adrenomedullin (ADM) antibody or an anti-adrenomedullin antibody fragment or anti-ADM non-Ig scaffold for use in prevention and therapy of dementia in a subject according to any of items 12 to 14, wherein the threshold level of mature ADM-NH₂ according to SEQ ID No.: 4 is equal to or below 15 pg/ml, preferably equal to or below 10 pg/ml, preferably equal or below 5 pg/ml.

16. Anti-adrenomedullin (ADM) antibody or an anti-adrenomedullin antibody fragment or anti-ADM non-Ig scaffold for use in prevention and therapy of dementia in a subject according to items 12 to 14, wherein the marker level ratio is in a range 0.2 to 0.75, preferably 0.3 to 0.6, preferably 0.4 to 0.5.

17. Anti-adrenomedullin (ADM) antibody or an anti-adrenomedullin antibody fragment or anti-ADM non-Ig scaffold for use in prevention and therapy of dementia in a subject according to items 11 to 16 wherein said subject is selected according to a method of item 10 and, wherein the sample of bodily fluid is selected from the group of blood, serum, plasma, urine, cerebrospinal fluid (CSF), and saliva.

18. Anti-adrenomedullin (ADM) antibody or an anti-adrenomedullin antibody fragment or anti-ADM non-Ig scaffold for use in prevention and therapy of dementia in a subject according to items 11 to 16, wherein at least one additional clinical parameter is determined selected from the group comprising age, race, mental status testing (e.g. mini-mental state examination (MMSE)), neuroimaging (CT, MRT, PET, SPECT), family history, ApoE4 genotype, Amyloidβ 1-42 (Aβ₁₋₄₂), Amyloidβ 1-40 (Aβ₁₋₄₀), total Tau-protein, phosphorylated Tau-protein (p-Tau 181, p-Tau 199, p-Tau 231).

19. Anti-adrenomedullin (ADM) antibody or an anti-adrenomedullin antibody fragment or anti-ADM non-Ig scaffold for use in prevention and therapy of dementia in a subject according to 12 to 18 items, wherein the level of said marker is determined by an immunoassay.

20. Anti-Adrenomedullin (ADM) antibody or an anti-ADM antibody fragment binding to adrenomedullin or anti-ADM non-Ig scaffold binding to adrenomedullin for use in prevention and therapy of dementia in a subject according to items 12-19, wherein said antibody or fragment or scaffold exhibits a binding affinity to ADM of at least 10⁻⁷ M.

21. Anti-ADM antibody or an anti-adrenomedullin antibody fragment or an anti-ADM non-Ig scaffold for use in prevention and therapy of dementia in a subject according to items 12-20, wherein said antibody or fragment or scaffold recognizes and binds to the N-terminal end (aa 1) of adrenomedullin.

22. Anti-ADM antibody or an anti-adrenomedullin antibody fragment or an anti-ADM non-Ig scaffold for use in prevention and therapy of dementia in a subject according to items 12-21, wherein said antibody or fragment or scaffold blocks the bioactivity of ADM not more than 80%, preferably not more than 50%.

### FIGURE DESCRIPTION

FIG. 1 shows a typical bio-ADM dose/signal curve and a bio-ADM dose signal curve in the presence of 100 μg/mL antibody NT-H.

FIG. 2 shows the bio-ADM concentrations in the MPP cohort and in an independent Alzheimer disease cohort

FIG. 3 shows a Kaplan-Meier-Plot of the bio-ADM concentrations in the MPP cohort for the prediction of Alzheimer's disease

FIG. 4 shows a Box-Plot of the bio-ADM concentrations in a subcohort of the MPP (case control) cohort for the prediction of Alzheimer's disease and in an independent AD cohort

FIG. 5 shows a Box-Plot of the MR-proADM concentrations in a subcohort of the MPP (case control) cohort for the prediction of Alzheimer's disease

FIG. 6 shows a Box-Plot of the bio-ADM/MR-proADM ratio in a subcohort of the MPP (case control) cohort for the prediction of Alzheimer's disease

FIG. 7 shows a ROC-Plot of bio-ADM (A) and the ratio of bio-ADM and MR-proADM (B) in a subcohort of the MPP (case control) cohort for the prediction of Alzheimer's disease

FIG. 8 shows the bio-ADM-values in healthy human individuals after administration of NT-H antibody.

### EXAMPLES

### Example 1

### Generation of Antibodies and Determination of Their Affinity Constants

Several human and murine antibodies were produced and their affinity constants were determined (see Table 1).

### Peptides/Conjugates for Immunization

Peptides for immunization were synthesized, see Table 1, (JPT Technologies, Berlin, Germany) with an additional N-terminal Cystein (if no Cystein is present within the selected ADM-sequence) residue for conjugation of the peptides to Bovine Serum Albumin (BSA). The peptides were covalently linked to BSA by using Sulfolink-coupling gel (Perbio Science, Bonn, Germany). The coupling procedure was performed according to the manual of Perbio.

The murine antibodies were generated according to the following method:

A Balb/c mouse was immunized with 100 μg Peptide-BSA-Conjugate at day 0 and 14 (emulsified in 100 μl complete Freund's adjuvant) and 50 μg at day 21 and 28 (in 100 μl incomplete Freund's adjuvant). Three days before the fusion experiment was performed, the animal received 50 μg of the conjugate dissolved in 100 μl saline, given as one intraperitoneal and one intra-venous injection.

Splenocytes from the immunized mouse and cells of the myeloma cell line SP2/0 were fused with 1 ml 50% polyethylene glycol for 30 s at 37° C. After washing, the cells were seeded in 96-well cell culture plates. Hybrid clones were selected by growing in HAT medium (RPMI 1640 culture medium supplemented with 20% fetal calf serum and HAT-Supplement). After two weeks the HAT medium is replaced with HT Medium for three passages followed by returning to the normal cell culture medium.

The cell culture supernatants were primary screened for antigen specific IgG antibodies three weeks after fusion. The positive tested microcultures were transferred into 24-well plates for propagation. After retesting, the selected cultures were cloned and recloned using the limiting-diluatoin technique and the isotypes were determined (see also Lane, R. D. 1985. J. Immunol. Meth. 81: 223-228; Ziegler et al. 1996. Horm. Metab. Res. 28: 11-15).

### Mouse Monoclonal Antibody Production

Antibodies were produced via standard antibody production methods (Marx et al, 1997. Monoclonal Antibody Production, ATLA 25, 121) and purified via Protein A. The antibody purities were >95% based on SDS gel electrophoresis analysis.

### Human Antibodies

Human Antibodies were produced by means of phage display according to the following procedure:

The human naive antibody gene libraries HAL7/8 were used for the isolation of recombinant single chain F-Variable domains (scFv) against ADM peptide. The antibody gene libraries were screened with a panning strategy comprising the use of peptides containing a biotin tag linked via two different spacers to the ADM peptide sequence. A mix of panning rounds using non-specifically bound antigen and streptavidin bound antigen were used to minimize background of non-specific binders. The eluted phages from the third round of panning have been used for the generation of monoclonal scFv expressing E. coli strains. Supernatant from the cultivation of these clonal strains has been directly used for an antigen ELISA testing (see also Hust et al. 2011. Journal of Biotechnology 152, 159-170; Schütte et al. 2009. PLoS One 4, e6625).

Positive clones have been selected based on positive ELISA signal for antigen and negative for streptavidin coated micro titer plates. For further characterizations the scFv open reading frame has been cloned into the expression plasmid pOPE107 (Hust et al. 2011. Journal of Biotechnology 152, 159-170), captured from the culture supernatant via immobilised metal ion affinity chromatography and purified by a size exclusion chromatography.

### Affinity Constants

To determine the affinity of the antibodies to ADM, the kinetics of binding of ADM to immobilized antibody was determined by means of label-free surface plasmon resonance using a Biacore 2000 system (GE Healthcare Europe GmbH, Freiburg, Germany). Reversible immobilization of the antibodies was performed using an anti-mouse Fc antibody covalently coupled in high density to a CMS sensor surface according to the manufacturer's instructions (mouse antibody capture kit; GE Healthcare) (Lorenz et al. 2011. Antimicrob Agents Chemother. 55(1): 165-173).

The monoclonal antibodies were raised against the below depicted ADM regions of human and murine ADM, respectively. The following table represents a selection of obtained antibodies used in further experiments. Selection was based on target region:

TABLE 1

| | | | | Affinity |
|---|---|---|---|---|
| Sequence | | ADM | Desi- | constants |
| Number | Antigen/Immunogen | Region | gnation | Kd (M) |
| SEQ ID: 21 | YRQSMNNFQGLRSFGCRFGTC | 1-21 | NT-H | 5.9 x 10⁻⁹ |
| SEQ ID: 22 | CTVQKLAHQIYQ | 21-32 | MR-H | 2 x 10⁻⁹ |
| SEQ ID: 23 | CAPRSKISPQGY-NH₂ | C-42- | CT-H | 1.1 x 10⁻⁹ |
| | | 52 | | |
| SEQ ID: 24 | YRQSMNQGSRSNGCRFGTC | 1-19 | NT-M | 3.9 x 10⁻⁹ |
| SEQ ID: 25 | CTFQKLAHQIYQ | 19-31 | MR-M | 4.5 x 10⁻¹⁰ |
| SEQ ID: 26 | CAPRNKISPQGY-NH₂ | C-40- | CT-M | 9 x 10⁻⁹ |
| | | 50 | | |

### Generation of Antibody Fragments by Enzymatic Digestion

The generation of Fab and F(ab)2 fragments was done by enzymatic digestion of the murine full-length antibody NT-M. Antibody NT-M was digested using a) the pepsin-based F(ab)2 Preparation Kit (Pierce 44988) and b) the papain-based Fab Preparation Kit (Pierce 44985). The fragmentation procedures were performed according to the instructions provided by the supplier. Digestion was carried out in case of F(ab)2-fragmentation for 8 h at 37° C. The Fab-fragmentation digestion was carried out for 16 h, respectively.

### Procedure for Fab Generation and Purification

The immobilized papain was equilibrated by washing the resin with 0.5 ml of Digestion Buffer and centrifuging the column at 5000×g for 1 minute. The buffer was discarded afterwards. The desalting column was prepared by removing the storage solution and washing it with digestion buffer, centrifuging it each time afterwards at 1000×g for 2 minutes. 0.5 ml of the prepared IgG sample where added to the spin column tube containing the equilibrated Immobilized Papain. Incubation time of the digestion reaction was done for 16 h on a tabletop rocker at 37° C. The column was centrifuged at 5000×g for 1 minute to separate digest from the Immobilized Papain. Afterwards the resin was washed with 0.5 ml PBS and centrifuged at 5000×g for 1 minute. The wash fraction was added to the digested antibody that the total sample volume was 1.0 ml. The NAb Protein A Column was equilibrated with PBS and IgG Elution Buffer at room temperature. The column was centrifuged for 1 minute to remove storage solution (contains 0.02% sodium azide) and equilibrated by adding 2 ml of PBS, centrifuge again for 1 minute and the flow-through discarded. The sample was applied to the column and resuspended by inversion. Incubation was done at room temperature with end-over-end mixing for 10 minutes. The column was centrifuged for 1 minute, saving the flow-through with the Fab fragments. (References: Coulter and Harris 1983. J. Immunol. Meth. 59, 199-203.; Lindner et al. 2010. Cancer Res. 70, 277-87; Kaufmann et al. 2010. PNAS. 107, 18950-5.; Chen et al. 2010. PNAS. 107, 14727-32; Uysal et al. 2009 J. Exp. Med. 206, 449-62; Thomas et al. 2009. J. Exp. Med. 206, 1913-27; Kong et al. 2009 J. Cell Biol. 185, 1275-840).

### Procedure for Generation and Purification of F(ab′)2 Fragments

The immobilized Pepsin was equilibrated by washing the resin with 0.5 ml of Digestion Buffer and centrifuging the column at 5000×g for 1 minute. The buffer was discarded afterwards. The desalting column was prepared by removing the storage solution and washing it with digestion buffer, centrifuging it each time afterwards at 1000×g for 2 minutes. 0.5 ml of the prepared IgG sample where added to the spin column tube containing the equilibrated Immobilized Pepsin. Incubation time of the digestion reaction was done for 16 h on a tabletop rocker at 37° C. The column was centrifuged at 5000×g for 1 minute to separate digest from the Immobilized Papain. Afterwards the resin was washed with 0.5 mL PBS and centrifuged at 5000×g for 1 minute. The wash fraction was added to the digested antibody that the total sample volume was 1.0 ml. The NAb Protein A Column was equilibrated with PBS and IgG Elution Buffer at room temperature. The column was centrifuged for 1 minute to remove storage solution (contains 0.02% sodium azide) and equilibrated by adding 2 mL of PBS, centrifuge again for 1 minute and the flow-through discarded. The sample was applied to the column and resuspended by inversion. Incubation was done at room temperature with end-over-end mixing for 10 minutes. The column was centrifuged for 1 minute, saving the flow-through with the Fab fragments. (References: Mariani et al. 1991. Mol. Immunol. 28: 69-77; Beale 1987. Exp Comp Immunol 11:287-96; Ellerson et al. 1972. FEBS Letters 24(3):318-22; Kerbel and Elliot 1983. Meth Enzymol 93:113-147; Kulkarni et al. 1985. Cancer Immunol Immunotherapy 19:211-4; Lamoyi 1986. Meth Enzymol 121:652-663; Parham et al. 1982. J Immunol Meth 53:133-73; Raychaudhuri et al. 1985. Mol Immunol 22(9):1009-19; Rousseaux et al. 1980. Mol Immunol 17:469-82; Rousseaux et al. 1983. J Immunol Meth 64:141-6; Wilson et al. 1991. J Immunol Meth 138:111-9).

### NT-H-Antibody Fragment Humanization

The antibody fragment was humanized by the CDR-grafting method (Jones et al. 1986. Nature 321, 522-525).

The following steps where executed to achieve the humanized sequence:
Total RNA extraction: Total RNA was extracted from NT-H hybridomas using the Qiagen kit.
First-round RT-PCR: QIAGEN® OneStep RT-PCR Kit (Cat No. 210210) was used. RT-PCR was performed with primer sets specific for the heavy and light chains. For each RNA sample, 12 individual heavy chain and 11 light chain RT-PCR reactions were set up using degenerate forward primer mixtures covering the leader sequences of variable regions. Reverse primers are located in the constant regions of heavy and light chains. No restriction sites were engineered into the primers.
Reaction Setup: 5× QIAGEN® OneStep RT-PCR Buffer 5.0 μl, dNTP Mix (containing 10 mM of each dNTP) 0.8 μl, Primer set 0.5 μl, QIAGEN® OneStep RT-PCR Enzyme Mix 0.8 μl, Template RNA 2.0 μl, RNase-free water to 20.0 μl, Total volume 20.0 μl PCR condition: Reverse transcription: 50° C., 30 mM; Initial PCR activation: 95° C., 15 mM Cycling: 20 cycles of 94° C., 25 sec; 54° C., 30 sec; 72° C., 30 sec; Final extension: 72° C., 10 mM Second-round semi-nested PCR: The RT-PCR products from the first-round reactions were further amplified in the second-round PCR. 12 individual heavy chain and 11 light chain RT-PCR reactions were set up using semi-nested primer sets specific for antibody variable regions.
Reaction Setup: 2×PCR mix 10 μl; Primer set 2 μl; First-round PCR product 8 μl; Total volume 20 μl; Hybridoma Antibody Cloning Report PCR condition: Initial denaturing of 5 min at 95° C.; 25 cycles of 95° C. for 25 sec, 57° C. for 30 sec, 68° C. for 30 sec; Final extension is 10 min 68° C.
After PCR is finished, run PCR reaction samples onto agarose gel to visualize DNA fragments amplified. After sequencing more than 15 cloned DNA fragments amplified by nested RT-PCR, several mouse antibody heavy and light chains have been cloned and appear correct. Protein sequence alignment and CDR analysis identifies one heavy chain and one light chain. As the amino acids on positions 26, 40 and 55 in the variable heavy chain and amino acid on position 40 in the variable light are critical to the binding properties, they may be reverted to the murine original. The resulting candidates are depicted below. (Padlan 1991. Mol. Immunol. 28, 489-498; Harris and Bajorath. 1995. Protein Sci. 4, 306-310).

Annotation for the antibody fragment sequences (SEQ ID No.: 13-22): bold and underline are the CDR 1, 2, 3 in chronologically arranged; italic are constant regions; hinge regions are highlighted with bold letters and the histidine tag with bold and italic letters.

(AM-VH-C)
SEQ ID No.: 13
QVQLQQSGAELMKPGASVKISCKATGYTFSRYWIEWVKQRPGHGLEWIGEILPGSG
STNYNEKFKGKATITADTSSNTAYMQLSSLTSEDSAVYYC [symbol] WGQGTTLT
VSSASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKRVEPK [symbol]
(AM-VH1)
SEQ ID No.: 14
QVQLVQSGAEVKKPGSSVKVSCKASGYTFSRYWISWVRQAPGQGLEWMGRILPGS
GSTNYAQKFQGRVTITADESTSTAYMELSSLRSEDTAVYYC [symbol] WGQGTTV
TVSSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPK [symbol]
(AM-VH2-E40)
SEQ ID No.: 15
QVQLVQSGAEVKKPGSSVKVSCKASGYTFSRYWIEWVRQAPGQGLEWMGRILPGS
GSTNYAQKFQGRVTITADESTSTAYMELSSLRSEDTAVYYC [symbol] WGQGTTV
TVSSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPK [symbol]
(AM-VH3-T26-E55)
SEQ ID No.: 16
QVQLVQSGAEVKKPGSSVKVSCKATGYTFSRYWISWVRQAPGQGLEWMGEILPGS
GSTNYAQKFQGRVTITADESTSTAYMELSSLRSEDTAVYYC [symbol] WGQGTTV
TVSSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPK [symbol]
(AM-VH4-T26-E40-E55)
SEQ ID No.: 17
QVQLVQSGAEVKKPGSSVKVSCKATGYTFSRYWIEWVRQAPGQGLEWMGEILPGS
GSTNYAQKFQGRVTITADESTSTAYMELSSLRSEDTAVYYC [symbol] WGQGTTV
TVSSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPK [symbol]
(AM-VL-C)
SEQ ID No.: 18
DVLLSQTPLSLPVSLGDQATISCRSSQSIVYSNGNTYLEWYLQKPGQSPKLLIYRVSN
RFSGVPDRFSGSGSGTDFTLKISRVEAEDLGVYYCFQGSHIPYTFGGGTKLEIKRTVA
APSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
(AM-VL1)
SEQ ID No.: 19
DVVMTQSPLSLPVTLGQPASISCRSSQSIVYSNGNTYLNWFQQRPGQSPRRLIYRVSN
RDSGVPDRFSGSGSGTDFTLKISRVEAEDVGVYYCFQGSHIPYTFGQGTKLEIKRTVA
APSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
(AM-VL2-E40)
SEQ ID No.: 20
DVVMTQSPLSLPVTLGQPASISCRSSQSIVYSNGNTYLEWFQQRPGQSPRRLIYRVSN
RDSGVPDRFSGSGSGTDFTLKISRVEAEDVGVYYCFQGSHIPYTFGQGTKLEIKRTVA
APSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC

### Example 2

### Development of an Immunoassay

An immunoassay was developed using antibodies generated against human ADM peptides (NT-H, MR-H and CT-H; see table 1).

Labelling procedure (tracer): 100 μg (100 μl) of antibody (1 mg/ml in PBS, pH 7.4) was mixed with 10 μl Akridinium NHS-ester (1 mg/ml in acetonitrile, InVent GmbH, Germany) (EP 0 353 971) and incubated for 20 mM at room temperature. Labelled CT-H was purified by Gel-filtration HPLC on Bio-Sil® SEC 400-5 (Bio-Rad Laboratories, Inc., USA). The purified labeled antibody was diluted in (300 mmol/L potassiumphosphate, 100 mmol/L NaCl, 10 mmol/L Na-EDTA, 5 g/L Bovine Serum Albumin, pH 7.0). The final concentration was approx. 800.000 relative light units (RLU) of labelled compound (approx. 20 ng labeled antibody) per 200 μL. Akridiniumester chemiluminescence was measured by using an AutoLumat LB 953 (Berthold Technologies GmbH & Co. KG).

Solid phase: Polystyrene tubes (Greiner Bio-One International AG, Austria) were coated (18 h at room temperature) with antibody (1.5 μg antibody/0.3 mL 100 mmol/L NaCl, 50 mmol/L TRIS/HCl, pH 7.8). After blocking with 5% bovine serum albumine, the tubes were washed with PBS, pH 7.4 and vacuum dried.

Calibrators: Synthetic human ADM (hADM) (Bachem, Switzerland) was linearily diluted using 50 mM Tris/HCl, 250 mM NaCl, 0.2% Triton X-100, 0.5% BSA, 20 tabs/L Protease Complete Protease Inhibitor Cocktail Tablets (Roche AG); pH 7.8. Calibrators were stored at −20° C. before use.

ADM Immunoassay: 50 μl of sample (or calibrator) was pipetted into coated tubes, after adding labeled second antibody (200 μl), the tubes were incubated for 2 h at room temperature. Unbound tracer was removed by washing 5 times (each 1 ml) with washing solution (20 mM PBS, pH 7.4, 0.1% Triton X-100). Tube-bound chemiluminescence was measured by using the LB 953 (Berthold Technologies GmbH & Co. KG). Antibodies were used in a sandwich immunoassay as coated tube and labeled antibody and combined in the following variations (see Table 2). Incubation was performed as described under hADM-Immunoassay. Results are given in ratio of specific signal (at 10 ng/ml ADM)/background (sample without ADM) signal.

TABLE 2

| Signal/noise ratio | NT-ADM tracer | MR-ADM tracer | CT-ADM tracer |
|---|---|---|---|
| NT-ADM | / | 195 | 241 |
| MR-ADM | 204 | / | 904 |
| CT-ADM | 260 | 871 | / |

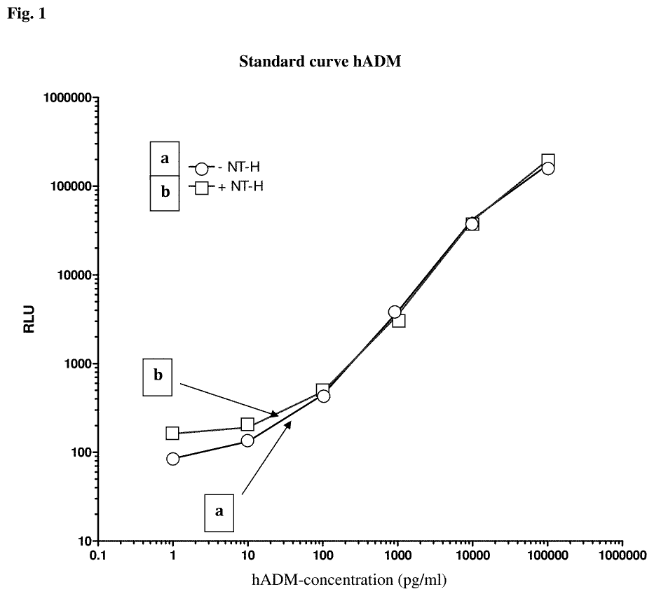

Surprisingly, we found the combination of MR-ADM and CT-ADM as combination for highest signal/noise ratio. Subsequently, we used this antibody-combination for further investigations to measure bio-ADM. We used anti-MR-ADM as solid phase antibody and anti-CT-ADM as labeled antibody. A typical dose/signal curve is shown in FIG. 1.

### Example 3

### Stability of Human Adrenomedullin

Human ADM was diluted in human Citrate plasma (n=5, final concentration 10 ng ADM/ml) and incubated at 24° C. At selected time points, aliquots were frozen at −20° C. Immediately after thawing the samples hADM was quantified by using the hADM immunoassay described above.

Table 3 shows the stability of hADM in human plasma at 24° C.

| Time | Average ADM | Relative loss of | Loss of immune |
|---|---|---|---|
| (h) | recovery (N = 5) | immune reactivity | reactivity %/h |
| 0 | 100 | / | / |
| 2 | 99.2 | 0.8 | 0.4 |
| 4 | 96.4 | 3.6 | 0.8 |
| 8 | 88.2 | 11.8 | 1.5 |
| | | | Average: 0.9%/h |

Surprisingly, using the antibody-combinations MR-ADM and CT-ADM in a sandwich immune assay, the pre-analytical stability of the analyte is high (only 0.9%/h average loss of immune reactivity). In contrast, using other assay methods, a plasma half-life of only 22 min was reported (Hinson et al. 2000 Endocrine Reviews 21(2):138-167). Since the time from taking sample to analysis in hospital routine is less than 2 h, the used ADM detection method is suitable for routine diagnosis. It is remarkable, that any non-routine additives to samples (like aprotinin, (Ohta et al. 1999. Clin Chem 45 (2): 244-251)) are not needed to reach acceptable ADM-immune reactivity stabilities.

### Example 4

### Reproducibility of Calibrator—Preparations and Interpreparation Variation of Calibrators

We found a high variation of results, preparing calibrators for ADM assays (average CV 8.5%, see Table 4). This may be due to high adsorption of hADM to plastic and glass surfaces (Lewis et al. 1998. Clinical Chemistry 44 (3): 571-577). This effect was only slightly reduced by adding detergents (up to 1% Triton X 100 or 1% Tween 20), protein (up to 5% BSA) and high ionic strength (up to 1M NaCl) or combinations thereof. Surprisingly, if a surplus of anti-NT-ADM antibody (10 μg/ml) is added to the calibrator dilution buffer, the recovery and reproducibility of ADM assay calibrator-preparations was substantially improved to <1% of inter preparation CV (Table 4). Coefficients of variation are given from 5 independent preparation runs. The calibrators were measured using the ADM assay described above (s/n-r =signal to noise ratio). For all following studies, we used an ADM assay, based on calibrators, prepared in the presence of 10 μg/ml of NT-ADM antibody and 10 μg/ml of NT-ADM antibody as supplement in the tracer buffer.

Fortunately, the presence of N-terminal antibodies did not affect the bio-ADM-signal generated by the combination of MR- and C-terminal antibodies (FIG. 1).

TABLE 4

| | In the presence | Inter | | Inter |
|---|---|---|---|---|
| | of NT-ADM | preparation | | preparation |
| | antibody | CV | Without | CV |
| | (10 μg/ml) | (%) | antibody | (%) |
| calibrator | | | | |
| 100 ng/ml | 3453 s/n-r | 0.9 | 2842 s/n-r | 2.8 |
| 10 ng/ml | 1946 s/n-r | 0.8 | 1050 s/n-r | 7.9 |
| 1 ng/ml | 179 s/n-r | 1.1 | 77 s/n-r | 14.8 |
| | | Average: 0.93 | | Average: 8.5 |

[symbol]

### Example 5

### Sensitivity

The goal of assay sensitivity was to completely cover the ADM concentration of healthy subjects and considerably lower concentrations.

### Bio-ADM Concentration in Healthy Subjects

Healthy subjects (n=88, 57 females, 31 males, mean age: 42.2 years) were measured using the bio-ADM assay (Weber et al. 2017. JALM, 2(2): 222-233). The median interquartile range (IQR) was 13.7 (9.6-18.7) pg/mL and mean (SD) was 15.6 (9.2) pg/mL. Since the assay sensitivity (limit of detection) was 3 pg/ml, 100% of healthy subjects were detectable using the described bio-ADM assay.

### Example 6

### Study Design and Population MPP

The Malmö Preventive Project (MPP) was funded in the mid-1970s to explore CV risk factors in general population, and enrolled 33,346 individuals living in Malmo (Fedorowski et al. 2010. Eur Heart J 31: 85-91). Between 2002 and 2006, a total of 18,240 original participants responded to the invitation (participation rate, 70.5%) and were screened including a comprehensive physical examination and collection of blood samples (Fava et al. 2013. Hypertension 2013; 61: 319-26). The re-examination in MPP is in the present study regarded as the baseline. Subjects with prior CVD at baseline were excluded. Bio-ADM was measured from plasma collected during baseline evaluation in 4,364 patients. 34 of the patients were already diagnosed as having Alzheimer's disease at the time of blood-sampling. A number of 187 patients developed AD within the following 7 years (incident AD). An informed consent was obtained from all participants and the Ethical Committee of Lund University, Lund, Sweden, approved the study protocol.

Information about dementia diagnoses was requested from the Swedish National Patient Register (SNPR). The diagnoses in the register were collected according to different revisions of the International Classification of Diseases (ICD) codes 290, 293 (ICD-8), 290, 331 (ICD-9) or F00, F01, F03, G30 (ICD-10). Since 1987, SNPR includes all in-patient care in Sweden and, in addition, contains data on outpatient visits including day surgery and psychiatric care from both private and public caregivers recorded after 2000. All-cause dementia was diagnosed according to the criteria of the Diagnostic and Statistical Manual of Mental Disorders (DSM)-III revised edition, whilst the DSM-IV criteria were applied for the Alzheimer's disease and vascular dementia diagnoses. Diagnoses were validated by a thorough review of medical records as well as neuroimaging data when available. A research physician assigned the final diagnosis for each patient and a geriatrician specialized in cognitive disorders was consulted in unresolved cases. We have chosen a data set for a case control study and MR-proADM was measured in a sub-cohort of MPP (n=250 controls and n=150 subjects with incident AD). Moreover, bio-ADM was measured in an independent cohort of patients that have already been diagnosed with Alzheimer's disease at the time of blood sampling (=prevalent AD; n=150).

A commercial fully automated homogeneous time-resolved fluoroimmunoassay was used to measure MR-proADM in plasma (BRAHMS MR-proADM KRYPTOR; BRAHMS GmbH, Hennigsdorf, Germany) (Caruhel et al. 2009. Clin Biochem. 42 (7-8):725-8).

Statistical analysis: Values are expressed as means and standard deviations, medians and interquartile ranges (IQR), or counts and percentages as appropriate. Group comparisons of continuous variables were performed using the Kruskal-Wallis test. Biomarker data were log-transformed. Cox proportional-hazards regression was used to analyse the effect of risk factors on survival in uni- and multivariable analyses. The assumptions of proportional hazard were tested for all variables. For continuous variables, hazard ratios (HR) were standardized to describe the HR for a biomarker change of one IQR. 95% confidence intervals (CI) for risk factors and significance levels for chi-square (Wald test) are given. The predictive value of each model was assessed by the model likelihood ratio chi-square statistic. The concordance index (C index) is given as an effect measure. It is equivalent to the concept of AUC adopted for binary outcome. For multivariable models, a bootstrap corrected version of the C index is given. Survival curves plotted by the Kaplan-Meier method were used for illustrative purposes. To test for independence of bio-ADM from clinical variables we used the likelihood ratio chi-square test for nested models.

All statistical tests were 2-tailed and a two-sided p-value of 0.05 was considered for significance. The statistical analyses were performed using R version 2.5.1 (http://www.r-project.org, library Design, Hmisc, ROCR) and Statistical Package for the Social Sciences (SPSS) version 22.0 (SPSS Inc., Chicago, Ill., USA).

### Results

Baseline characteristics of the cohort are shown in table 5.

| Variable | n = 4364 |
|---|---|
| Age | 69.4 (6.2) |
| gender male | 3008 (68.9%) |
| Current Smoking | 835 (19.1%) |
| AHT | 1476 (33.8%) |
| HDL | 1.4 (0.4) |
| LDL | 3.7 (1.0) |
| BMI | 27.1 (6.2) |
| SBP | 146.6 (20.3) |
| prevalent Diabetes | 466 (10.7%) |
| Incident AD | 187 (4.3%) |
| Prevalent AD | 34 (0.8%) |

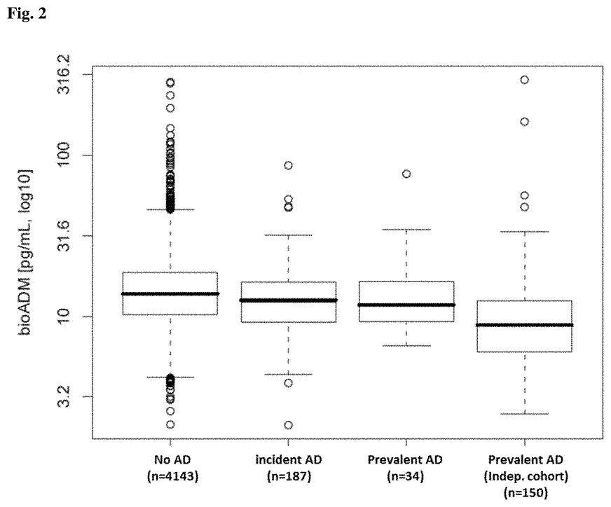

The bio-ADM concentrations in the MPP cohort and in an independent Alzheimer disease cohort are shown in FIG. 2. The bio-ADM concentrations in patients developing AD over time (incident AD) and in patients with AD at the time of blood sampling in the independent cohort (prevalent AD) are significantly lower compared to the non-AD group (p=0.01 and <0.0001, respectively). The group with prevalent AD from the MPP cohort (n=34) also presented with lower bio-ADM concentrations compared to the non-AD group but was not statistically significant, which is due to the small sample size.

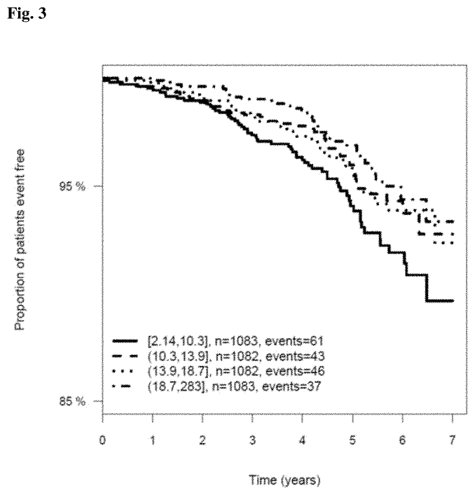

Low bio-ADM plasma concentration strongly predicts Alzheimer's disease with a Hazard Ratio (HR) of 0.73 (CI 0.6-0.87; p<0.001). FIG. 3 shows a Kaplan-Meier Plot for the prediction of Alzheimer's disease with bio-ADM concentrations (prevalent AD cases were excluded from the analysis). The lowest quartile is associated with the highest risk of getting AD.

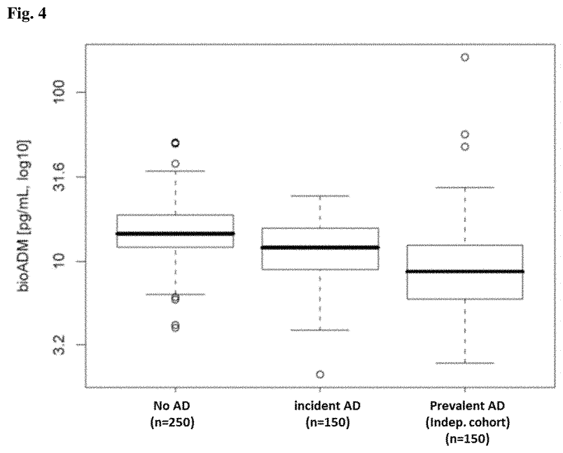

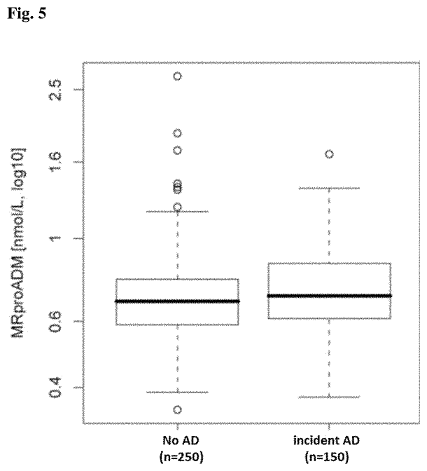

We created a data set for case-control choosing 400 patients from the MPP cohort (free from prior CVD and AD) with n=250 subjects who did not develop AD and n=150 subjects who did develop AD within the follow-up time of 7 years. Again, bio-ADM concentrations were significantly lower (p<0.0001 for all comparisons) in patients with incident AD and in patients with prevalent AD (independent cohort) compared to the non-AD group (FIG. 4). In addition, MR-proADM was measured in the MPP case control subjects and was slightly but not significantly higher in incident AD compared to the non-AD group (FIG. 5).

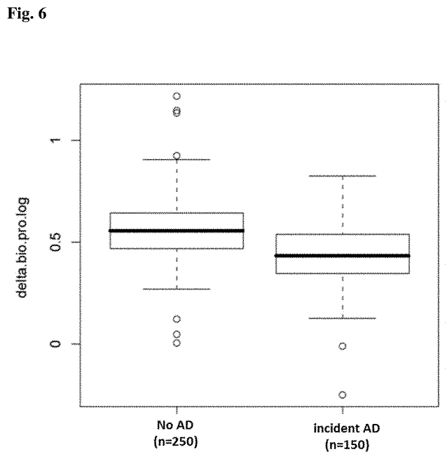

In a next step, we combined both biomarkers, bio-ADM and MR-proADM. For the calculation of the ratio, the concentration of the two markers has to be preferably expressed in the same unit (e.g. pmol/L). Therefore, in terms of calculating the ratio, concentrations for bio-ADM were calculated in pmol/L. The ratio of bio-ADM and MR-proADM is significantly decreased in subjects with incident AD when compared to non-AD subjects (p<0.0001; FIG. 6). Bio-ADM alone strongly predicts Alzheimer's disease with an Odds Ratio (OR) of 0.44 (CI 0.33-0.58). However, the ratio of bio-ADM and MR-proADM is better than bio-ADM alone (p<0.001) with an OR of 0.32 (CI 0.23-0.44). The respective receiver operating curves (ROC plots) for bio-ADM and the ratio of bio-ADM and MR-proADM are shown in

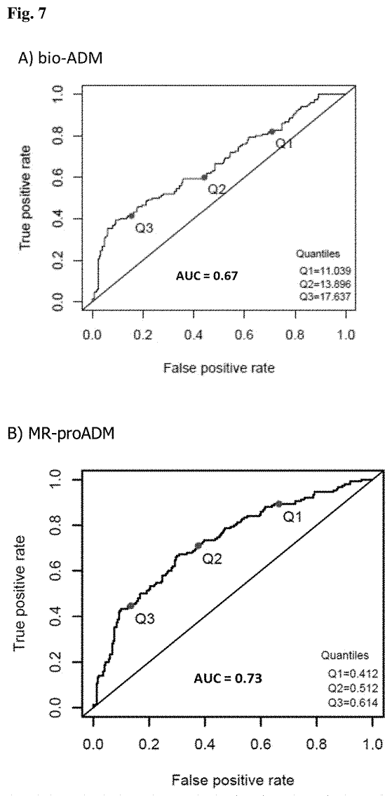

FIGS. 7 A and B, respectively and revealed an AUC of 0.67 (95% CI 0.61-0.72) for bio-ADM and of 0.73 (95% CI 0.68-0.78) for the ratio between bio-ADM and MR-proADM. Morover, both, bio-ADM and the ratio of bio-ADM and MR-proADM are independent of age and gender.

### Example 7—Administration of NT-H in Healthy Humans

The study was conducted in healthy male subjects as a randomized, double-blind, placebo-controlled, study with single escalating doses of NT-H antibody administered as intravenous (i.v.) infusion in 3 sequential groups of 8 healthy male subjects each (1st group 0.5 mg/kg, 2nd group 2 mg/kg, 3rd group 8 mg/kg) of healthy male subjects (n=6 active, n=2 placebo for each group).

The main inclusion criteria were written informed consent, age 18-35 years, agreement to use a reliable way of contraception and a BMI between 18 and 30 kg/m².

Subjects received a single i.v. dose of NT-H antibody (0.5 mg/kg; 2 mg/kg; 8 mg/kg) or placebo by slow infusion over a 1-hour period in a research unit.

The baseline bio-ADM-values in the 4 groups did not differ. Median bio-ADM values were 7.1 pg/mL in the placebo group, 6.8 pg/mL in the first treatment group (0.5 mg/kg), 5.5 pg/mL in second treatment group (2 mg/kg) and 7.1 pg/mL in the third treatment group (8 mg/mL).

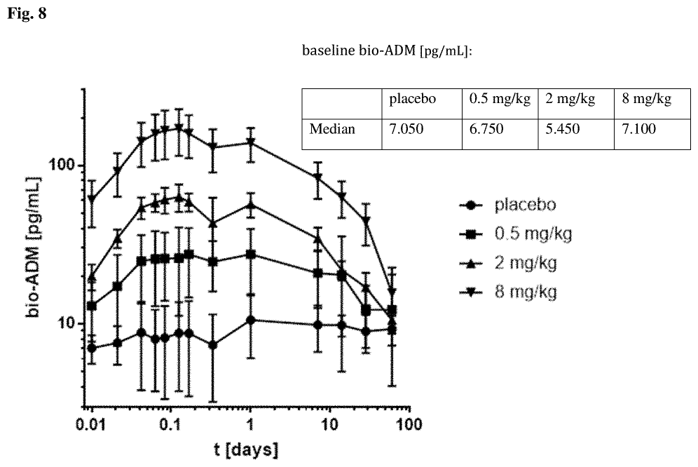

The results show, that bio-ADM-values rapidly increased within the first 1.5 hours after administration of NT-H antibody in healthy human individuals, then reached a plateau and slowly declined (FIG. 8).

1. A method for:
a) diagnosing dementia, or
b) determining the risk of getting dementia in a subject that does not have dementia, or
c) monitoring therapy or monitoring or guiding intervention in a subject that has dementia, or
d) monitoring therapy or monitoring or guiding preventive intervention in a subject that is at risk of getting dementia,
wherein the level of mature ADM-NH₂ according to SEQ ID No.: 4 is determined in a sample of bodily fluid of a subject and wherein said level of mature ADM-NH₂ is compared with a threshold level,
and wherein
a) said subject is diagnosed with dementia if the level of mature ADM-NH₂ according to SEQ ID No.: 4 is below said threshold level, or wherein
b) said subject has an enhanced risk of getting dementia if said level of mature ADM-NH₂ according to SEQ ID No.: 4 is below said threshold level, or wherein
c) the status of a subject having dementia or being at risk of getting dementia is improving under therapy or intervention if said level of mature ADM-NH₂ according to SEQ ID No.: 4 is increased during the course of therapy or intervention and/or wherein intervention maybe continued if said level of mature ADM-NH₂ according to SEQ ID No.: 4 is increased above said threshold.

2. A method for:
a) diagnosing dementia, or
b) determining the risk of getting dementia in a subject that does not have dementia, or
c) monitoring therapy or monitoring or guiding intervention in a subject that has dementia, or
d) monitoring preventive therapy or monitoring or guiding preventive intervention in a subject that is at risk of getting dementia,
wherein a marker ratio is determined that maybe ratio of the level of mature ADM-NH₂ according to SEQ ID No.: 4 determined in a sample of bodily fluid of said subject to the level of pro-Adrenomedullin or a fragment thereof (which is not mature ADM-NH₂ according to SEQ ID No.: 4) determined in a sample of bodily fluid of said subject
and wherein said fragment of pro-Adrenomedullin is selected from a group comprising PAMP: SEQ ID No. 2, MR-proADM: SEQ ID No. 3, ADM-Gly: SEQ ID No.: 5 and CT-proADM: SEQ ID No. 6
and wherein said marker ratio is compared to a threshold, ratio
and wherein
a) said subject is diagnosed with dementia if the marker ratio of mature ADM-NH₂/pro-Adrenomedullin or a fragment thereof is below said ratio threshold, or wherein
b) said subject has an enhanced risk of getting dementia if the marker ratio of mature ADM-NH₂/pro-Adrenomedullin or a fragment thereof is below said ratio threshold, or wherein
c) the status of a subject having dementia or being at risk of getting dementia is improving under therapy or intervention if said marker ratio is increased during the course of therapy or intervention
or alternatively to the above marker ratio the level of mature ADM-NH₂ according to SEQ ID No.: 4 is determined in a sample of bodily fluid of said subject and the level of pro-Adrenomedullin or a fragment thereof (which is not mature ADM-NH₂ according to SEQ ID No.: 4) is determined in a sample of bodily fluid of said subject and both determined level are combined in a mathematical algorithm, wherein the result of said mathematical algorithm is used for diagnosing dementia, or determining the risk of getting dementia in a subject that does not have dementia, or monitoring therapy or monitoring or guiding intervention in a subject that has dementia, or monitoring preventive therapy or monitoring or guiding preventive intervention in a subject that is at risk of getting dementia.

3. A method according to claim 1, wherein the threshold level of mature ADM-NH₂ according to SEQ ID No.: 4 is equal or below 15 pg/ml, preferably equal or below 10 pg/ml, preferably equal or below 5 pg/ml.

4. A method according to claim 2, wherein the ratio threshold is in a range of 0.2 to 0.75, preferably 0.3 to 0.6, preferably 0.4 to 0.5.

5. A method according to claim 1, wherein said sample is selected from the group of blood, serum, plasma, urine, cerebrospinal fluid (CSF), and saliva.

6. A metohd according to claim 1, wherein the sample of bodily fluid is taken from a subject that has never had a diagnosis of dementia or MCI at the time of sample taking.

7. A method according to claim 1, wherein said method is used for patient stratification to select a patient for treatment with an Anti-adrenomedullin (ADM) antibody or an anti-adrenomedullin antibody fragment or anti-ADM non-Ig scaffold for use in prevention and therapy of dementia in a subject, wherein said anti-ADM antibody or anti-ADM fragment or anti-ADM non-Ig scaffold binds to the N-terminal part (aa 1-21) of adrenomedullin:
(SEQ ID No. 21)
YRQSMNNFQGLRSFGCRFGTC.

8. Anti-adrenomedullin (ADM) antibody or an anti-adrenomedullin antibody fragment or anti-ADM non-Ig scaffold for use in prevention and therapy of dementia in a subject, wherein said anti-ADM antibody or anti-ADM fragment or anti-ADM non-Ig scaffold binds to the N-terminal part (aa 1-21) of adrenomedullin:
(SEQ ID No. 21)
YRQSMNNFQGLRSFGCRFGTC.

9. Anti-adrenomedullin (ADM) antibody or an anti-adrenomedullin antibody fragment or anti-ADM non-Ig scaffold for use in prevention and therapy of dementia in a subject according to claim 8, wherein said subject has a level of mature ADM-NH₂ according to SEQ ID No.: 4 determined in a sample of bodily fluid of said subject below a threshold level and/or has a marker ratio that is the ratio of the level of mature ADM-NH₂ according to SEQ ID No.: 4 determined in a sample of bodily fluid of said subject to the level of pro-Adrenomedullin or a fragment thereof determined in a sample of bodily fluid of said subject and wherein said marker level ratio is below a ratio threshold,

10. Anti-adrenomedullin (ADM) antibody or an anti-adrenomedullin antibody fragment or anti-ADM non-Ig scaffold for use in prevention and therapy of dementia in a subject according to claim 9 wherein, said fragment of pro-Adrenomedullin is selected from a group comprising PAMP (SEQ ID No. 2), MR-proADM (SEQ ID No. 3), ADM-Gly (SEQ ID No.: 5) and CT-proADM (SEQ ID No. 6).

11. Anti-adrenomedullin (ADM) antibody or an anti-adrenomedullin antibody fragment or anti-ADM non-Ig scaffold for use in prevention and therapy of dementia in a subject according to claim 10, wherein said subject has a level of mature ADM-NH₂ according to SEQ ID No.: 4 determined in a sample of bodily fluid of said subject below a threshold level and/or has a marker ratio that is the ratio of the level of mature ADM-NH₂ according to SEQ ID No.: 4 determined in a smaple of bodily fluid of said subjecxt to the level of pro-Adrenomedullin or a fragment thereof dteermined in a sample of bodily fluid of said subject and wherein said marker level ratio is below a ratio threshold.

12. Anti-adrenomedullin (ADM) antibody or an anti-adrenomedullin antibody fragment or anti-ADM non-Ig scaffold for use in prevention and therapy of dementia in a subject according to claim 9, wherein the threshold level of mature ADM-NH₂ according to SEQ ID No.: 4 is equal to or below 15 pg/ml, preferably equal to or below 10 pg/ml, preferably equal or below 5 pg/ml.

13. Anti-adrenomedullin (ADM) antibody or an anti-adrenomedullin antibody fragment or anti-ADM non-Ig scaffold for use in prevention and therapy of dementia in a subject according to claim 9, wherein the marker level ratio is in a range 0.2 to 0.75, preferably 0.3 to 0.6, preferably 0.4 to 0.5. Anti-adrenomedullin (ADM) antibody or an anti-adrenomedullin antibody fragment or anti-ADM non-Ig scaffold for use in prevention and therapy of dementia in a subject according to claim 9 and, wherein the sample of bodily fluid is selected from the group of blood, serum, plasma, urine, cerebrospinal fluid (CSF), and saliva.


SEQUENCE LISTING (29 sequences)

SEQ ID NO: 1 - protein, 185 aa, Homo sapiens
MKLVSVALMYLGSLAFLGADTARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 2 - protein, 20 aa, Homo sapiens
ARLDVASEFRKKWNKWALSR

SEQ ID NO: 3 - protein, 48 aa, Homo sapiens
ELRMSSSYPTGLADVKAGPAQTLIRPQDMKGASRSPEDSSPDAARIRV

SEQ ID NO: 4 - protein, 52 aa, Homo sapiens
YRQSMNNFQGLRSFGCRFGTCTVQKLAHQIYQFTDKDKDNVAPRSKISPQGY

SEQ ID NO: 5 - protein, 53 aa, Homo sapiens
YRQSMNNFQGLRSFGCRFGTCTVQKLAHQIYQFTDKDKDNVAPRSKISPQGYG

SEQ ID NO: 6 - protein, 38 aa, Homo sapiens
RRRRRSLPEAGPGRTLVSSKPQAHGAPAPPSGSAPHFL

SEQ ID NO: 7 - protein, 8 aa, Artificial Sequence
  Description of Artificial Sequence Synthetic peptide
  Adrezizumab
GYTFSRYW

SEQ ID NO: 8 - protein, 8 aa, Artificial Sequence
  Description of Artificial Sequence Synthetic peptide
  Adrezizumab
ILPGSGST

SEQ ID NO: 9 - protein, 11 aa, Artificial Sequence
  Description of Artificial Sequence Synthetic peptide
  Adrezizumab
TEGYEYDGFDY

SEQ ID NO: 10 - protein, 11 aa, Artificial Sequence
  Description of Artificial Sequence Synthetic peptide
  Adrezizumab
QSIVYSNGNTY

SEQ ID NO: 11 - protein, 3 aa, Artificial Sequence
  Description of Artificial Sequence Synthetic peptide
  Adrezizumab
RVS

SEQ ID NO: 12 - protein, 9 aa, Artificial Sequence
  Description of Artificial Sequence Synthetic peptide
  Adrezizumab
FQGSHIPYT

SEQ ID NO: 13 - protein, 225 aa, Artificial Sequence
  Description of Artificial Sequence Synthetic polypeptide
  Adrezizumab
QVQLQQSGAELMKPGASVKISCKATGYTFSRYWIEWVKQRPGHGLEWIGEILPGSGSTNYNEKFKGKATITADTSSNTAYMQLSSLTSEDSAVYYCTEGYEYDGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKHHHHHH

SEQ ID NO: 14 - protein, 225 aa, Artificial Sequence
  Description of Artificial Sequence Synthetic polypeptide
  Adrezizumab
QVQLVQSGAEVKKPGSSVKVSCKASGYTFSRYWISWVRQAPGQGLEWMGRILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKHHHHHH

SEQ ID NO: 15 - protein, 225 aa, Artificial Sequence
  Description of Artificial Sequence Synthetic polypeptide
  Adrezizumab
QVQLVQSGAEVKKPGSSVKVSCKASGYTFSRYWIEWVRQAPGQGLEWMGRILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKHHHHHH

SEQ ID NO: 16 - protein, 225 aa, Artificial Sequence
  Description of Artificial Sequence Synthetic polypeptide
  Adrezizumab
QVQLVQSGAEVKKPGSSVKVSCKATGYTFSRYWISWVRQAPGQGLEWMGEILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKHHHHHH

SEQ ID NO: 17 - protein, 225 aa, Artificial Sequence
  Description of Artificial Sequence Synthetic polypeptide
  Adrezizumab
QVQLVQSGAEVKKPGSSVKVSCKATGYTFSRYWIEWVRQAPGQGLEWMGEILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKHHHHHH

SEQ ID NO: 18 - protein, 219 aa, Artificial Sequence
  Description of Artificial Sequence Synthetic polypeptide
  Adrezizumab
DVLLSQTPLSLPVSLGDQATISCRSSQSIVYSNGNTYLEWYLQKPGQSPKLLIYRVSNRFSGVPDRFSGSGSGTDFTLKISRVEAEDLGVYYCFQGSHIPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 219 aa, Artificial Sequence
  Description of Artificial Sequence Synthetic polypeptide
  Adrezizumab
DVVMTQSPLSLPVTLGQPASISCRSSQSIVYSNGNTYLNWFQQRPGQSPRRLIYRVSNRDSGVPDRFSGSGSGTDFTLKISRVEAEDVGVYYCFQGSHIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 219 aa, Artificial Sequence
  Description of Artificial Sequence Synthetic polypeptide
  Adrezizumab
DVVMTQSPLSLPVTLGQPASISCRSSQSIVYSNGNTYLEWFQQRPGQSPRRLIYRVSNRDSGVPDRFSGSGSGTDFTLKISRVEAEDVGVYYCFQGSHIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 21 aa, Homo sapiens
YRQSMNNFQGLRSFGCRFGTC

SEQ ID NO: 22 - protein, 12 aa, Homo sapiens
CTVQKLAHQIYQ

SEQ ID NO: 23 - protein, 12 aa, Homo sapiens
CAPRSKISPQGY

SEQ ID NO: 24 - protein, 19 aa, Mus musculus
YRQSMNQGSRSNGCRFGTC

SEQ ID NO: 25 - protein, 12 aa, Mus musculus
CTFQKLAHQIYQ

SEQ ID NO: 26 - protein, 12 aa, Mus musculus
CAPRNKISPQGY

SEQ ID NO: 27 - protein, 14 aa, Homo sapiens
YRQSMNNFQGLRSF

SEQ ID NO: 28 - protein, 10 aa, Homo sapiens
YRQSMNNFQG

SEQ ID NO: 29 - protein, 6 aa, Homo sapiens
YRQSMN